(19) United States
(12) Patent Application Publication
(10) Pub. No.: US 2004/0110191 A1
(43) Pub. Date: Jun. 10, 2004

(54) Comparative analysis of nucleic acids using population tagging

(76) Inventors: Matthew M. Winkler, Austin, TX (US); David Brown, Austin, TX (US)

Correspondence Address: FULBRIGHT & JAWORSKI L.L.P., 600 CONGRESS AVE., SUITE 2400, AUSTIN, TX, 78701, US

(21) Appl. No.: 10/632,539

(22) Filed: Jul. 31, 2003

Related U.S. Application Data
(63) Continuation of application No. PCT/US02/03097, filed on Jan. 31, 2002.
(63) Continuation of application No. PCT/US02/03168, filed on Jan. 31, 2002.
(63) Continuation of application No. PCT/US02/02892, filed on Jan. 31, 2002.
(63) Continuation of application No. PCT/US02/03169, filed on Jan. 31, 2002.
(60) Provisional application No. 60/265,694, filed on Jan. 31, 2001.
(60) Provisional application No. 60/265,693, filed on Jan. 31, 2001.
(60) Provisional application No. 60/265,695, filed on Jan. 31, 2001.
(60) Provisional application No. 60/265,692, filed on Jan. 31, 2001.

(51) Int. Cl. C12Q 1/68; C12P 19/34

(52) U.S. Cl. 435/6; 435/91.2

(57) ABSTRACT

Disclosed are methods that allow one or more nucleic acid targets to be compared across two or more nucleic acid samples. Nucleic acid tags are appended to the samples to be assessed, such that each sample has a unique tag. The tagged nucleic acids are then mixed, and the targets within the mixture are amplified. The amplification products are distinguished using the unique tag domains to reveal the abundance of the amplification products derived from each sample, which correlates to the relative abundance of the target in the samples.

This patent application claims priority to U. S. Provisional Patent Application No. 60/265,694.

The present application was filed concurrently with: PCT Application No. ______ on Jan. 31, 2002, entitled “METHODS FOR NUCLEIC ACID FINGERPRINT ANALYSIS,” which claims priority to U.S. Provisional Patent Application No. 60/265,693, filed on Jan. 31, 2001, PCT Application No. ______ filed Jan. 31, 2002, entitled “COMPETITIVE POPULATION NORMALIZATION FOR COMPARATIVE ANALYSIS OF NUCLEIC ACID SAMPLES,” which claims priority to U. S. Provisional Patent Application No. 60/265,695 filed on Jan. 31, 2001; and PCT Application No. ______, filed Jan. 31, 2002 entitled “COMPETITIVE AMPLIFICATION OF FRACTIONATED TARGETS FROM MULTIPLE NUCLEIC ACID SAMPLES,” which claims priority to U.S. Provisional Patent Application No. 60/265,692, filed on Jan. 31, 2001. The disclosure of each of the above-identified applications is specifically incorporated herein by reference in its entirety without disclaimer.

### BACKGROUND OF THE INVENTION

1. Field of the Invention

The present invention relates generally to the fields of nucleic acid amplification. More particularly, it concerns using nucleic acid amplification to compare two or more nucleic acid populations. The present invention incorporates methods for adding nucleic acid tag sequences to nucleic acid populations to promote amplification and differentiation of one or more nucleic acid targets present in the nucleic acid population(s).

2. Description of Related Art

Gene expression analysis is the study of how much protein gets synthesized in a cell or tissue from a defined set of genes. The identity and abundance of proteins in a sample determines the type and state of the cell, tissue, organ or organism from which it derived. Unfortunately, the quantitative assessment of many different proteins in a given biological sample is exceedingly difficult and requires large amounts of sample.

The identity and relative abundance of RNAs in a sample can reveal which proteins are being expressed in a biological sample and at what levels. The study of RNA expression is often easier than that of protein expression, thus RNA analysis is preferred by investigators studying the dynamics of gene expression.

Techniques commonly used for RNA expression analysis can be divided into those aimed at quantifying one or a few RNA targets in a sample and those designed to screen a large number of RNA targets in a sample. Techniques for analyzing one or a few RNA targets include Northern blotting, nuclease protection assay, relative RT-PCR, and competitive RT-PCR. Techniques for analyzing many targets simultaneously are differential display and array analysis.

a. Northern Analysis

Northern blots are used extensively for assaying the expression of one or a few mRNAs within RNA samples. Northern blots are produced by fractionating mRNA or other RNA populations by gel electrophoresis and then transferring and crosslinking the RNAs to an appropriate solid support. Northern blots are analyzed using target specific probes. Probes are generally labeled RNA or DNA molecules possessing sequences complementary to genes that are being studied. The probes are incubated with the blot and hybridization occurs between probe and complementary target sequences. Unhybridized probe is removed by washing and the bound molecules are detected using autoradiography or an equivalent method.

Absolute quantification of a given target can be achieved by including a sense strand control in the blot to provide correlation of hybridization signal to target concentration. In addition to being used for RNA expression analysis, Northern blots provide the size of the gene transcript, the existence of alternative splice variants of the gene, and the presence of closely related genes.

Northern blot analysis has three shortcomings. First, the method is labor intensive. The process of fractionating RNA samples, transferring to membranes, generating probes for analysis, hybridizing probe to the Northern blot, and detecting hybridized probe requires several days to complete and numerous independent reagents. Second, Northern blot analysis is incapable of detecting rare messages. In general, 100,000 to 1,000,000 target molecules must be present in a sample for it to be detected via northern blotting. This tends to limit Northern blotting to the analysis of moderately and highly abundant RNA targets. Third, the method is typically limited to detecting a single target per hybridization reaction. For multiple targets to be assessed in a single hybridization experiment, the desired RNA targets must be of significantly different sizes and similar abundance. These two criteria are rarely met by multiple RNA targets.

b. Nuclease Protection Assay

Another method of RNA expression analysis is the nuclease protection assay. There are two types of nuclease protection assay, the S1 assay and the ribonuclease protection assay (RPA), which differ primarily in the nuclease used to digest the samples being assayed (Sambrook, 1989). The S1 Assay uses Nuclease S1 while RPA typically uses RNase A and/or RNase T1. Both methods use labeled nucleic acid probes that are complementary to specific RNA targets in a sample. The labeled probes are incubated with RNA samples to allow hybridization to occur between the target RNA and labeled probe. The mixture is then treated with one or more of the nucleases described above, each of which specifically degrades single-stranded RNA and/or DNA. Any labeled probe that is not hybridized to target RNA is degraded, leaving only the hybridized probe. The undigested probe is fractionated by gel electrophoresis and visualized. The signal from the undigested probe can be quantified to determine the amount of target RNA in the samples being assessed.

Because the labeled probes used for nuclease protection assays can be of any size, the technique is extremely effective for simultaneously analyzing multiple RNA targets. Probes of differing sizes for multiple target RNAs can be mixed, incubated with a RNA sample, digested, and fractionated to provide quantitative data on several different targets. However nuclease protection assays are limited to relatively abundant RNA targets. As with Northern blot analysis, RPA does not incorporate target amplification or probe signal amplification and is therefore limited to the study of RNA that is present in at least about 10,000 copies per sample.

c. Relative RT-PCR

Reverse transcription-polymerase chain reaction (RT-PCR) is a method for RNA analysis that incorporates nucleic acid amplification to allow exceedingly rare RNA targets to be characterized. The most commonly applied method of RNA expression analysis incorporating RT-PCR is Relative RT-PCR. Relative RT-PCR provides a reasonably accurate estimate of the relative abundance of a particular target RNA between multiple samples. The method involves reverse transcribing and amplifying a given target in multiple samples using identical primers and other amplification reagents. The amplification products for each sample are fractionated by gel electrophoresis in adjacent lanes and the intensity of the product band resulting from amplification of each sample is compared. The intensity of the target amplification product correlates with the abundance of the target in the original sample, providing a relative measure of the target in each of the samples. Relative RT-PCR is most accurate when an effective internal control RNA is co-amplified with the RNA target to normalize the RNA samples.

Relative RT-PCR is far more sensitive than Northern analysis and nuclease protection assays. In addition, the technique is easier to set up than the above methods because no probes need be synthesized for analysis. However, the technique requires a great deal of effort to ensure that the amplification reaction is in linear range at the point that the amplification products are assessed. In addition, the method is only relatively quantitative which means that it can help determine if a particular transcript is present at greater or lesser levels in one sample compared to another. However, relative RT-PCR cannot reliably quantify the difference in the amount of RNA present in two samples.

d. Competitive RT-PCR

Competitive RT-PCR can accurately quantify transcripts from a single gene in single sample populations. The method makes use of known concentrations of an exogenous RNA standard, known as a competitor, added to an RNA sample prior to reverse transcription. The competitor is amplified by the same primers as the endogenous target. Provided the competitor and endogenous targets are amplified at the same rate and yield products that can be readily distinguished, the concentration of the endogenous target in the sample RNA can be accurately determined. When the amplification products from the endogenous and exogenous RNA targets are equal, the concentrations of the competitor and RNA target are equal in the starting reaction. Because the concentration-of the competitor RNA is known, the concentration of the endogenous target in the sample may be determined.

In a typical experiment, equal amounts of an RNA sample are aliquotted into tubes with differing amounts of competitor. The RNA/competitor mixtures are reverse transcribed and amplified with primers specific to the target and competitor. The mixture that results in equal amounts of amplification product for both the target and competitor reveals the concentration of the target in the sample.

Competitive RT-PCR suffers from four drawbacks. First, a competitor must be synthesized, quantified, and tested for each target RNA being assessed. This requires a substantial outlay of time and effort on the part of the practitioner. Second, each sample being assessed is typically aliquoted into multiple reactions with varying quantities of competitor to provide a standard curve against which the RNA target can be accurately quantified. Using multiple reactions to assess each sample is costly both in terms of reagents and time. Where limited samples are being analyzed, this can be a serious limitation. Third, only single targets can be assessed in each set of reactions due to problems with amplifying multiple targets with multiple primers in a single reaction. The second and third drawbacks conspire to limit the number of targets that can be characterized per sample. Fourth, only single samples can be assessed in each set of reactions because the amplification products from one sample cannot be distinguished from the amplification products from a second sample.

e. Adaptor-Tagged Competitive-PCR

Adaptor-Tagged Competitive-PCR (ATAC-PCR) is a variation of the competitive RT-PCR procedure that reduces the requirement for competitor synthesis and increases the number of samples that can be assessed in a single reaction (Kato 1997, European Patent Application #98302726). ATAC-PCR makes one sample population a competitor for another sample population. ATAC-PCR accomplishes this by converting mRNA samples to double-stranded cDNA using a reverse transcriptase, digesting the cDNA samples with a restriction enzyme, and ligating adapters to members of the cDNA samples at their respective restriction sites. The adapters share a primer binding site but differ in size or sequence (i.e., unique restriction or hybridization sites). The adapter-tagged cDNAs are mixed and amplified with a gene-specific primer and a PCR primer specific to the shared adapter sequence present at the proximal ends of the cDNA populations. If the adapters used for tagging were different sizes, then the amplification products resulting from PCR are directly assessed by gel electrophoresis. If the adapters from the populations differ by a restriction site, then the amplification products are aliquoted into different restriction digestion reactions to cleave the tag sequences from amplification products derived from specific samples. The digestion products are then assessed by gel electrophoresis. Because the amplification products generated from each sample population are different sizes, they can be readily fractionated and quantified. The ratio of amplification products generated from each sample reflects the relative abundance of the target in each sample.

ATAC-PCR has four shortcomings. First, four steps are required to convert an RNA sample to a population that is ready for PCR amplification. If any of these steps vary between the samples being compared, inaccuracies will result. Thus inefficient or biased reverse transcription, second strand cDNA synthesis, restriction digestion, or adapter ligation can profoundly affect the data being generated. Second, ATAC-PCR initiates amplification with double-stranded nucleic acids that all possess a domain that is complementary to the adapter-specific primer. Therefore, target and non-target sequences are at least linearly amplified from the amplification domain of the adapter. This generates background that can affect quantitative analysis. Third, ATAC-PCR is apparently limited to the comparative analysis of targets in only a few samples. The ATAC-PCR patent and subsequent uses of the technology (Matoba 2000) describe its use to quantify single targets in up to three sample populations. This is apparently due to limitations in resolving more than three amplification products using the size differences possible with ligated adapters. Fourth, only a single target is being assessed in each amplification reaction. This is a burden on both the time required to assess a reasonable number of target sequences and the amount of cDNA sample required to accommodate a reasonable number of amplification reactions.

f. Differential Display

Welsh and McClelland (1990) were the first to report that PCR using low temperature annealing conditions with arbitrary primers reproducibly generate a collection of distinct amplification products from a nucleic acid sample. They referred to the pattern of bands as a fingerprint and used the fingerprints of different samples to identify RNAs that were present at different levels in the samples. A number of techniques were developed to identify differentially expressed transcripts that incorporated arbitrary priming and fingerprint analysis.

The most popular technique employing nucleic acid fingerprint analysis is Differential Display-Reverse Transcription-PCR (DD-RT-PCR). The general procedure is described in U.S. Pat. No. 5,262,311. An oligonucleotide with a polydT sequence with at least one non-dT residue at its 3′ end, called an anchored oligodT primer, is used to prime reverse transcription of a eukaryotic RNA population. The resulting cDNA is amplified by PCR using the same anchored oligodT primer used for reverse transcription and one or more primers of 9 to 20 nucleotides possessing some arbitrary sequence(s). The amplified products from different samples are typically displayed by gel electrophoresis. Those bands that are unique or appear to be of different signal intensities between two samples should represent unique or differentially expressed genes. They are generally excised from the gel, cloned, and sequenced.

The primary problem associated with differential display is the high rate of false positives that occur with the technique. U.S. Pat. No. 5,712,126 estimates that approximately 80% of the amplification products that appear to be differentially expressed in a DD-RT-PCR experiment turn out not to differ in relative expression level. U.S. Pat. No. 5,712,126 also indicates that when a single RNA sample is split and the two resulting samples are taken through the DD-RT-PCR procedure, the fingerprint patterns differ by 5%. The inconsistency in generating fingerprints has kept the technique from becoming a preferred method for comparing RNA or DNA samples.

g. Gene Array Analysis

Gene arrays are solid supports upon which a collection of gene-specific probes has been spotted at defined locations. The probes localize complementary labeled targets from a nucleic acid sample via hybridization. One of the most common uses for gene arrays is the comparison of the global expression patterns of different mRNA populations. A typical experiment involves isolating RNA from two or more tissue or cell samples. The RNAs are reverse transcribed using labeled nucleotides and target specific, oligodT, or random-sequence primers to create labeled cDNA populations. The cDNAs are denatured from the template RNA and hybridized to identical arrays. The hybridized signal on each array is detected and quantified. The signal emitting from each gene-specific spot is compared between the populations. Genes expressed at different levels in the samples generate different amounts of labeled cDNA and this results in spots on the array with different amounts of signal.

The direct conversion of RNA populations to labeled cDNAs is widely used because it is simple and largely unaffected by enzymatic bias. However, direct labeling requires large quantities of RNA to create enough labeled product for moderately rare targets to be detected by array analysis. Most array protocols recommend that 2.5 μg of polyA or 50 μg of total RNA be used for reverse transcription (Duggan 1999). For practitioners unable to isolate this much RNA from their samples, global amplification procedures have been used.

The most often cited of these global amplification schemes is antisense RNA (aRNA) amplification (U.S. Pat. Nos. 5,514,545 and 5,545,522, Phillips 1996). aRNA amplification involves reverse transcribing RNA samples with an oligo-dT primer that has a transcription promoter such as the T7 RNA polymerase consensus promoter sequence at its 5′ end. First strand reverse transcription creates single-stranded cDNA. Following first strand cDNA synthesis, the template RNA that is hybridized to the cDNA is partially degraded creating RNA primers. The RNA primers are then extended to create double-stranded DNAs possessing transcription promoters. The population is transcribed with an appropriate RNA polymerase to create an RNA population possessing sequence from the cDNA. Because transcription results in tens to thousands of RNAs being created from each DNA template, substantive amplification can be achieved. The RNAs can be labeled during transcription and used directly for array analysis, or unlabeled aRNA can be reverse transcribed with labeled dNTPs to create a cDNA population for array hybridization. In either case, the detection and analysis of labeled targets is the same as described above.

Although aRNA amplification provides a way to assess small RNA samples, it is not yet clear that the amplification scheme is appropriate for comparative analysis. One potential problem is that amplification may be biased. An amplification bias is a disproportionate amplification of the individual mRNA species in a given population. Amplification bias will alter the levels of target sequences in one population in ways that are unlikely to be maintained in a second population. This will lead to array data that suggest that some genes are differentially expressed between two populations when in actuality the differences merely result from different amplification rates for those targets between the two populations. This problem is not unique to aRNA amplification. In fact, aRNA amplification is used by researchers performing gene array analysis because it is the least problematic of the methods used for nucleic acid amplification.

The methods that currently exist for comparing the levels of RNA in different samples suffer either from an inability to detect rare messages (e.g., Northern and RPA analysis) or suffer from irreproducibility of amplification products. For most of the techniques employing amplification, the populations being compared are assessed separately so that amplification products from each sample can be readily distinguished. In DD-RT-PCR, for example, the RNA populations being compared are amplified in different reaction vessels and assessed by electrophoresis in adjacent lanes on an acrylamide gel.

Unfortunately, nucleic acid amplification is notoriously non-quantitative. Slight variations in the amplification efficiency of different reactions can lead to significant differences in the amount of amplification product that is generated from even identical nucleic acid samples. Amplification efficiency is dependent on many factors, including enzyme, nucleotide, and primer concentration; reaction temperature; and the makeup of the nucleic acid population being assessed. Slight variations in any of these components can induce differential amplification between different nucleic acid samples and suggest that target(s) within the samples are present at different levels when in fact that may not be true.

The variation in amplification efficiency derives largely from an inability to generate identical reaction conditions in two distinct vessels. The only way to achieve identical amplification efficiencies is to perform amplification in a single reaction. Amplifying nucleic acids from different samples would require that the amplification products generated from each sample be distinguishable following amplification. To date, no robust methods for achieving this have been developed.

### SUMMARY OF THE INVENTION

The present invention overcomes the limitations of the art by providing methods for co-amplifying and characterizing one or more nucleic acid targets in two or more nucleic acid samples. The invention involves appending sequences to the RNA or DNA comprising a nucleic acid sample. The appended sequences are identical for all members of one sample and unique for each sample being assessed. These unique sequences, also referred to as “tags,” can comprise any of a number of different types of domains and be appended to the target nucleic acid sequences in any of a variety of ways. The differentially tagged samples are mixed and targets within the sample mixture are amplified. The amplification products derived from targets in each sample are distinguished using the unique tag sequences appended to the targets from each sample prior to amplification.

In a broad aspect, the invention relates to methods of comparing one or more nucleic acid targets within two or more samples, comprising:

a) appending at least a first nucleic acid tag comprising at least a first amplification domain and at least a first differentiation domain to at least a first nucleic acid target of at least a first sample;

b) appending at least a second nucleic acid tag comprising at least a second amplification domain and at least a second differentiation domain to the first nucleic acid target of at least a second sample, wherein the second differentiation domain is different than the first differentiation domain;

c) amplifying said first nucleic acid target of the first sample and said first nucleic acid target of the second sample, wherein said amplifying produces at least a first amplified nucleic acid comprising at least the first differentiation domain and a segment of the target nucleic acid from the first sample and at least a second amplified nucleic acid comprising at least the second differentiation domain and a segment of the target nucleic acid from the second sample;

d) differentiating the first amplified nucleic acid from the second amplified nucleic acid; and

e) comparing abundance of the differentiated nucleic acid from the first nucleic acid target of said first sample to abundance of the differentiated nucleic acid from the first nucleic acid target of said second sample.

In presently preferred cases, the amplification will involve co-amplification of the first target nucleic acid and the second target nucleic acid in the same reaction mixture.

It is important to recognize that the present invention is useful for determining the abundance of a target nucleic acid in a sample, and that this encompasses the practice of the methods disclosed herein even when a target nucleic acid that is being assayed for is not present in a given sample. For example, it is possible that the target may be missing from a first sample, but present in a second sample in a given procedure. If this is the case, then it will not be possible to append a tag to the target in the first sample or, to amplify the target in the first sample. Therefore, the differentiation procedure will result in a determination that there was target present in the second sample, but not in the first. It is, therefore, not necessary that a target be present in any given sample for assays employing the methods disclosed herein to be within the scope of the invention.

In many applications, the nucleic acid target and/or the nucleic acid tag will be single-stranded nucleic acid. However this in not required in all embodiments of the invention, and those of skill will be able to follow the teachings of the specification to employ double-stranded nucleic acids in the invention. The nucleic acid target can be an RNA, DNA or a combination thereof. It is not required that the nucleic acid target be of natural origin, and the target can contain synthetic nucleotides. In specific aspects, the nucleic acid target is an RNA, for example, prokaryotic or eukaryotic RNA, total RNA, polyA RNA, an in vitro RNA transcript or a combination thereof. In other facets, the nucleic acid target may comprise DNA, such as, for example, cDNA, genomic DNA or a combination thereof. In certain aspects, at least one of the samples comprises nucleic acid isolated from a biological sample from, for example, a cell, tissue, organ or organism. In other aspects, at least one of the samples may comprise nucleic acid from an environmental sample. Of course, there is no need for all of the samples compared in a particular assay to be of the same source or type of source. A single sample may contain nucleic acid from a single source, or it may be the result of combining nucleic acids from multiple sources.

While, at its most basic level, there can be only one nucleic acid of interest in the samples, the advantages of the invention allow one to analyze a variety of nucleic acid targets in the samples at the same time. Therefore, in many instances, the first nucleic acid target will be only one of a plurality of nucleic acid targets to be analyzed in the samples. For example, the. techniques disclosed herein and in co-pending U.S. patent application Ser. No. 60/265,694, entitled “METHODS FOR NUCLEIC ACID FINGERPRINT ANALYSIS,” filed on Jan. 31, 2001; U.S. patent application Ser. No. 60/265,692, entitled “COMPETITIVE POPULATION NORMALIZATION FOR COMPARATIVE ANALYSIS OF NUCLEIC ACID SAMPLES,” filed on Jan. 31, 2001; and U.S. patent application Ser. No. 60/265,695 entitled “COMPETITIVE AMPLIFICATION OF FRACTIONATED TARGETS FROM MULTIPLE NUCLEIC ACID SAMPLES,” filed on Jan. 31, 2001, allow for many samples to be compared at once.

Further, while, at the most basic level, the methods of the invention may be employed with only two samples, in many cases, the first and second sample are two samples of a plurality of samples. One of the advantages of the invention is the ability of it to be used to analyze. many samples simultaneously. In preferred embodiments, the tags used for each sample will comprise a differentiation domain that is unique to that sample.

Of course, in cases where there are a plurality of samples, there will typically be a plurality of tags. Those of skill in the art will be able to employ the teachings of this specification to prepare appropriate tags. Typically, the number of unique tags required for a given procedure will be equal to the number of samples to be analyzed.

In presently preferred embodiments of the invention, the differentiation domains of the tags are appended between the nucleic acid target sequence and the amplification domain. In this manner, the differentiation domain is assured of being amplified during the amplification process, and is present in the amplified nucleic acid. Of course, those of skill in the art will realize that there are other positions of the differentiation and amplification domains in tags, and will be able to utilize tags with the domains in a variety of functional positions.

The amplification domains of nucleic acid tags may comprise any appropriate sequences as described elsewhere in the specification or known to those of skill in the art. In some preferred embodiments, the amplification domain comprises a primer binding domain and/or a transcription domain. In many cases, the amplification domains are the same for all targets being assessed in a given sample. However, in some embodiments the amplification domains could be. specific for a nucleic acid target. In preferred embodiments, the amplification domain for a first nucleic acid sample will be functionally equivalent to the amplification domain of a second sample and functionally equivalent to any amplification domains of any other samples. As used in this manner, “functionally equivalent” means that the amplification domains provide amplification of the target nucleic acid in the same manner and at the same rate. In the simplest embodiments of the invention, the amplification domain for a first nucleic acid target of a first sample will be identical to the amplification domains of the same target in any other samples.

The differentiation domains useful in the invention can be of any form described elsewhere in this specification or apparent to those of skill in view of the specification. In preferred embodiments, the differentiation domain will comprise at least a primer binding domain, a transcription domain, a size differentiation domain, an affinity domain, a unique sequence domain, or a restriction enzyme domain. For embodiments that involve differentiating amplification products by synthesizing labeled nucleic acids, all of the tags employed to label amplification products from one or a plurality of targets in a given sample will have functionally equivalent and/or identical differentiation domains, which domains are distinct from the differentiation domains used to label the amplification products of other samples. Further, in these embodiments of the invention, all samples assayed in the same protocol are labeled with the same type of differentiation domains, i.e. all are labeled with a primer binding domain or a transcription domain rather than different samples in the same protocol being labeled with different types of differentiation domains. Of course, those of skill will recognize that it is possible to use different types of differentiation domains in the same protocol, it is just not presently preferred.

In some embodiments, the differentiation domains are primer binding domains. In this case, differentiating comprises binding a first primer to at least one segment of each primer binding domain, and performing a primer extension reaction. Under this version of the invention, there will usually be as many primer extension reactions as there were, samples, each run on a different aliquot of co-amplified nucleic acid. This is because each sample will have a unique primer binding domain as its differentiation domain, and the result of each primer extension reaction will be to produce differentiated nucleic acid specific to each sample from the amplification products. In many cases the resulting differentiated nucleic acid is labeled with a detectable moiety, according to methods discussed elsewhere in the specification.

In other embodiments, differentiation domains are transcription domains, and in some even more specific embodiments, the differentiation domain comprises a promoter for a prokaryotic RNA polymerase. In these embodiments, differentiating comprises at least one transcription reaction. Typically, there will be as many such reactions as there were samples mixed for comparative analysis, with each reaction involving an aliquot of co-amplified nucleic acid. In most cases the differentiated nucleic acid will include a detectable moiety.

There are a variety of methods described herein and/or known to those of skill which will allow for the differentiation of the first amplified nucleic acid from the second amplified nucleic acid. While many of these comprise production of at least one differentiated nucleic acid from the first or second amplified nucleic acid, others involve distinguishing the amplification products directly.

The differentiation domains can be size differentiation domains, and, in this case, differentiating comprises distinguishing the amplification products by size. Alternatively, the differentiation domains may be restriction enzyme cleavage domains. If the differentiation domain is a restriction enzyme domain, differentiation can comprise cleaving a restriction enzyme cleavage site to promote the ligation of a label or at least one additional domain to a segment of a nucleic acid tag, or, alternatively, cleaving the restriction enzyme site to remove a label. A plurality of samples may be assessed using size differentiation domains or restriction enzyme cleavage domains.

In other embodiments, the differentiation domains are unique sequence domains and differentiating comprises sequencing through the differentiation domains of the amplified nucleic acids.

In other embodiments, the differentiation domains are affinity domains and differentiation comprises binding at least a first ligand to at least a segment of the affinity domain. Such a ligand may comprise a nucleic acid, or other type of ligand disclosed herein. The ligands employed in the invention may be labeled, and in some cases, the binding of a ligand to the affinity domain will result in production of a detectable signal. The ligands used in these embodiments of the invention may be bound to a solid support, for example, a membrane, a bead, a glass slide, an array, or a microtiter well. Support-bound ligands may be used to separate the amplified nucleic acid targets into fractions according to the sample from which the target derives.

In some embodiments of the invention, the nucleic acid tags may further comprise at least one additional domain of the type described elsewhere in the specification, for example, a labeling domain, a restriction enzyme domain, a secondary amplification domain, a secondary differentiation domain or a sequencing primer binding domain.

Some specific methods of the invention comprise comparing one or more nucleic acid targets within two or more samples, comprising:

a) appending at least a first nucleic acid tag comprising at least a first amplification domain and at least a first differentiation domain to at least a first nucleic acid target of at least a first sample, wherein said first differentiation domain comprises at least one affinity domain, primer binding domain, or transcription domain;

b) appending at least a second nucleic acid tag comprising at least a second amplification domain and at least a second differentiation domain to the first nucleic acid target of at least a second sample, wherein the second differentiation domain is different than the first differentiation domain and comprises at least one affinity domain, primer binding domain, or transcription domain;

c) co-amplifying said first nucleic acid target of the first sample and said first nucleic acid target of the second sample, wherein said amplifying produces at least a first amplified nucleic acid comprising at least the first differentiation domain and a segment of the target nucleic acid from the first sample and at least a second amplified nucleic acid comprising at least the second differentiation domain and a segment of the target nucleic acid from the second sample;

d) differentiating the first amplified nucleic acid from the second amplified nucleic acid; and

e) comparing abundance of the differentiated nucleic acid from the first nucleic acid target of said first sample to abundance of the differentiated nucleic acid from the first nucleic acid target of said second sample.

Other specifically preferred embodiments comprise comparing one or more nucleic acid targets within two or more samples, comprising:

a) appending at least a first nucleic acid tag comprising a first amplification domain and a first differentiation domain to at least a first nucleic acid target of at least a first sample, wherein the first differentiation domain comprises a first transcription domain, and wherein the differentiation domain of the first tag is appended between the first nucleic acid target sequence and the amplification domain;

b) appending at least a second nucleic acid tag comprising a second amplification domain and a second differentiation domain to the first nucleic acid target of at least a second sample, wherein the second differentiation domain comprises a second transcription domain that is different than the first transcription domain, and wherein the differentiation domain of the second tag is appended between the at least a first nucleic acid target sequence and the amplification domain;

c) co-amplifying the first nucleic acid target of the first sample and the first nucleic acid target of the second sample, wherein the amplifying produces at least a first amplified nucleic acid comprising the at least first transcription domain and a segment of the target nucleic acid from the first sample and a second amplified nucleic acid comprising at least the second transcription domain and a segment of the target nucleic acid from the second sample;

d) differentiating the first amplified nucleic acid, wherein the differentiating comprises transcription from the first transcription domain to produce at least a first differentiated nucleic acid;

e) differentiating the second amplified nucleic acid, wherein the differentiating further comprises transcription from the second transcription domain to produce at least a second differentiated nucleic acid; and

f) comparing abundance of the differentiated nucleic acid from the first nucleic acid target of said first sample to abundance of the differentiated nucleic acid from the first nucleic acid target of said second sample.

Additionally, in some aspects, the invention relates to methods of comparing one or more nucleic acid targets within two or more samples, comprising:

a) appending at least a first nucleic acid tag comprising a first amplification domain and a first differentiation domain to at least a first nucleic acid target of at least a first sample, wherein the first differentiation domain comprises a first primer binding domain, and wherein the differentiation domain of the first tag is appended between the first nucleic acid target sequence and the amplification domain;

b) appending at least a second nucleic acid tag comprising a second amplification domain and a second differentiation domain to the first nucleic acid target of at least a second sample, wherein the second differentiation domain comprises a second primer binding domain that is different than the first primer binding domain, and wherein the differentiation domain of the second tag is appended between the at least a first nucleic acid target sequence and the amplification domain;

c) co-amplifying the first nucleic acid target of the first sample and the first nucleic acid target of the second sample, wherein the amplifying produces at least a first amplified nucleic acid comprising at least the first primer binding domain and a segment of the target nucleic acid and a second amplified nucleic acid from the first sample comprising at least the second primer binding domain and a segment of the target nucleic acid from the second sample;

d) differentiating the first amplified nucleic acid, wherein the differentiating comprises annealing at least a first differentiation primer to the first primer binding domain, wherein the differentiating further comprises extension of the first differentiation primer to produce at least a first differentiated nucleic acid;

e) differentiating the second amplified nucleic acid, wherein the differentiating further comprises annealing at least a second differentiation primer to the second primer binding domain, wherein the differentiating further comprises extension of the second differentiation primer to produce at least a second differentiated nucleic acid; and

f) comparing abundance of the differentiated nucleic acid from the first nucleic acid target of the first sample to abundance of the differentiated nucleic acid from the first nucleic acid target of the second sample.

Other specific embodiments involve comparing one or more single-stranded nucleic acid targets within two or more samples, comprising:

a) appending at least a first single-stranded nucleic acid tag comprising a first amplification domain and a first differentiation domain to at least a first nucleic acid target of at least a first sample, wherein the first differentiation domain comprises a first size differentiation domain, and wherein the differentiation domain of the first tag is appended between the first nucleic acid target sequence and the amplification domain;

b) appending at least a second single-stranded nucleic acid tag comprising a second amplification domain and a second differentiation domain to the first nucleic acid target of at least a second sample, wherein the second differentiation domain comprises a second size differentiation domain that is different than the first size differentiation domain, and wherein the differentiation domain of the second tag is appended between the at least a first nucleic acid target sequence and the amplification domain;

c) co-amplifying the first nucleic acid target of the first sample and the first nucleic acid target of the second sample, wherein the co-amplifying produces at least a first amplified nucleic acid comprising at least the first size differentiation domain and a segment of the target nucleic acid and a second amplified nucleic acid comprising at least the second size differentiation domain and a segment of the target nucleic acid;

d) differentiating the first amplified nucleic acid, wherein said differentiating comprises determining the electrophoretic mobility of the first amplified nucleic acid;

e) differentiating the second amplified nucleic acid, wherein said differentiating further comprises determining the electrophoretic mobility of the second amplified nucleic acid; and

f) comparing abundance of the differentiated nucleic acid from the first nucleic acid target of said first sample to abundance of the differentiated nucleic acid from the first nucleic acid target of said second sample.

Other embodiments involve, comparing one or more nucleic acid targets within two or more samples, comprising:

a) appending at least a first nucleic acid tag comprising a first amplification domain and a first differentiation domain to at least a first nucleic acid target of at least a first sample, wherein the first differentiation domain comprises a first affinity domain, and wherein the differentiation domain of the first tag is appended between the first nucleic acid target sequence and the amplification domain;

b) appending at least a second nucleic acid tag comprising a second amplification domain and a second differentiation domain to the first nucleic acid target of at least a second sample, wherein the second differentiation domain comprises a second affinity domain that is different than the first affinity domain, and wherein the differentiation domain of the second tag is appended between the at least a first nucleic acid target sequence and the amplification domain;

c) co-amplifying the first nucleic acid target of the first sample and the first nucleic acid target of the second sample to produce at least a first amplified nucleic acid comprising at least the first affinity domain and a segment of the target nucleic acid from the first sample and a second amplified nucleic acid comprising at least the second affinity domain and a segment of the target nucleic acid from the second sample;

d) differentiating the first amplified nucleic acid, wherein the differentiating comprises binding of the first amplified nucleic acid to an at least a first ligand;

f) differentiating the second amplified nucleic acid, wherein the differentiating further comprises binding of the second amplified nucleic acid to an at least a second ligand; and

g) comparing abundance of the differentiated nucleic acid from the first nucleic acid target of said first sample to abundance of the differentiated nucleic acid from the first nucleic acid target of said second sample.

In most embodiments described above, the amplification domains will be at least functionally equivalent, and often, identical. Furthermore, differentiation is probably achieved using the differentiation domains.

As used herein in the specification, “a” or “an” may mean one or more. As used herein in the claim(s), when used in conjunction with the word “comprising”, the words “a” or “an” may mean one or more than one. As used herein “another” may mean at least a second or more. As used herein, a “plurality” means “two or more.”

As used herein, “plurality” means more than one. In certain specific aspects, a plurality may mean 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 400, 500, 750, 1,000, 2,000, 3,000, 4,000, 5,000, 7,500, 10,000, 15,000, 20,000, 30,000, 40,000, 50,000, 60,000, 70,000, 80,000, 90,000, 100,000, 125,000, 150,000, 200,000 or more, and any integer derivable therein, and any range derivable therein.

As used herein, “any integer derivable therein” means a integer between the numbers described in the specification, and “any range derivable therein” means any range selected from such numbers or integers.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. A general schematic for population tagging.

FIG. 2. Schematic for tagged nucleic acid targets.

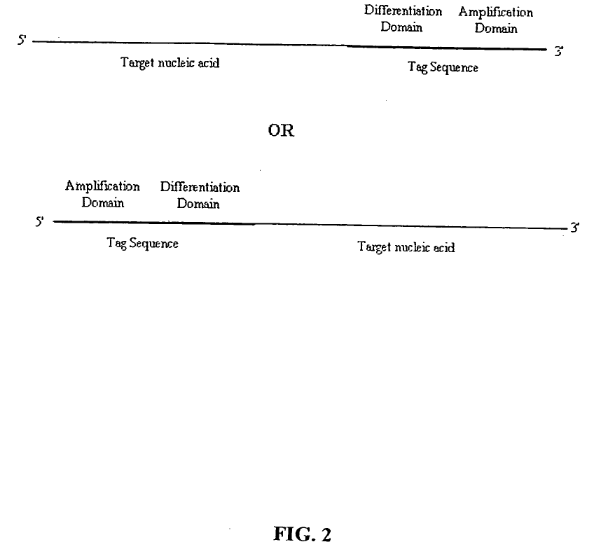

FIG. 3. Schematic showing differential labeling of amplified samples by primer extension.

FIG. 4. Schematic showing differential labeling of amplified samples by transcription.

FIG. 5. Differentiation of amplified samples by affinity isolation.

FIG. 6. Quantitative analysis using size differentiation domains.

FIG. 7. Competitive display.

FIG. 8. Schematic for tagged array analysis.

FIG. 9. Schematic for massively parallel sample analysis of single target.

### DESCRIPTION OF ILLUSTRATIVE EMBODIMENTS

In certain embodiments, the present invention provides simple procedures for directly comparing single or multiple nucleic acid targets in two or more samples. By a process called “population tagging,” tags are appended to RNA or DNA populations. The tag sequences are different for each nucleic acid population being analyzed. In all embodiments, the differentially tagged nucleic acids are mixed and the resulting mixed sample is applied to one of a variety of procedures that comprises amplification of target(s) in the sample.

In all embodiments, the amplified population is analyzed by using the unique tag sequences of the RNA or DNA samples to reveal the relative abundance of amplification products that derive from each of the nucleic acid samples. In certain embodiments, the analysis comprises the synthesis of a differentiated population of nucleic acids for analysis. In other embodiments, the amplification products are directly assessed in a way that distinguishes products with unique tag sequences. The present invention incorporates competitive amplification as do other techniques. However, the invention is superior to these techniques due to its stream-lined approach and multiplex potential.

For instance, unlike competitive PCR, the present invention does not require that a competitor be synthesized and accurately quantified prior to quantitative analysis. This greatly reduces the effort required to quantify target nucleic acids. Competitive RT-PCR involves amplifying mixtures of sample and competitor in multiple reactions for each sample being assessed. The present invention allows multiple samples to be mixed and amplified in a single reaction, improving the throughput of expression analysis and decreasing costs associated with each sample. The present invention can be readily used to quantify multiple known targets in multiple samples or even screen unknown targets in samples. In comparison, competitive RT-PCR is used exclusively to quantify single targets in single samples.

The invention differs from ATAC-PCR in several manners. In preferred embodiments, the present invention requires only a single step to tag a nucleic acid population. This reduces the likelihood that inaccuracies will result from variable reaction efficiencies. In contrast, ATAC-PCR requires four independent enzymatic reactions to tag a nucleic acid population which greatly increases the chances of sample-to-sample variability that can create quantitative aberrations in the experimental data. In preferred embodiments of the invention, tagged nucleic acids are single-stranded and require the action of a target specific primer to initiate amplification. In contrast, ATAC-PCR initiates amplification with double-stranded nucleic acids that all possess a domain that is complementary to the adapter-specific primer. Therefore, target and non-target sequences are at least linearly amplified from the amplification domain of the adapter. This generates background that is not found using the single-stranded material that initiates amplification in preferred aspects of the present invention. In certain embodiments of the invention, analysis of differentiated populations do not rely upon differences in the size(s) of amplification products. Thus, the methods of the present invention may analyze or compare a virtually unlimited number of samples in a single amplification reaction. In contrast, ATAC-PCR suffers functional limitations due to its reliance upon size to differentiate amplification products from different samples. The methods of the present invention may be used to quantify multiple known targets in multiple samples or even screen unknown targets in samples. In contrast, ATAC-PCR is described for use to quantify single known targets in up to three samples.

A. Nucleic Acids: Tags and Samples

Embodiments of the present invention involve nucleic acids in many forms. Nucleic acid samples are collections of RNA and/or DNA derived or extracted from chemical or enzymatic reactions, biological samples, or environmental samples. Nucleic acid tags are nucleic acids of a defined sequence that are appended to nucleic acids in a sample to facilitate its analysis. There are many potential types of tags for use in the invention, which are described elsewhere in this specification.

1. General Description of Nucleic Acids

The general term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.” The term “oligonucleotide” refers to a molecule of between 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84,.85, 86, 87, 88, 89, 90, 91, 92, 93, 94, 95, 96, 97, 98, 99, and 100 nucleobases in length, and any range derivable therein. The term “polynucleotide” refers to at least one molecule of greater than about 100 nucleobases in length.

a. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in a manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, a purine or pyrimidine substituted by one or more of an alkyl, caboxyalkyl, amino, hydroxyl, halogen (i.e., fluoro, chloro, bromo, or iodo), thiol or alkylthiol moeity. Preferred alkyl (e.g., alkyl, caboxyalkyl, etc.) moeities comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. A table of non-limiting purine and pyrimidine derivatives and analogs is also provided herein below.

TABLE 1
Purine and Pyrmidine Derivatives or Analogs

| Abbr. | Modified base description | Abbr. | Modified base description |
|---|---|---|---|
| Ac4c | 4-acetylcytidine | Mam5s2u | 5-methoxyaminomethyl-2-thiouridine |
| Chm5u | 5-(carboxyhydroxylmethyl) uridine | Man q | Beta,D-mannosylqueosine |
| Cm | 2′-O-methylcytidine | Mcm5s2u | 5-methoxycarbonylmethyl-2-thiouridine |
| Cmnm5s2u | 5-carboxymethylamino-methyl-2-thioridine | Mcm5u | 5-methoxycarbonylmethyluridine |
| Cmnm5u | 5-carboxymethylaminomethyluridine | Mo5u | 5-methoxyuridine |
| D | Dihydrouridine | Ms2i6a | 2-methylthio-N6-isopentenyladenosine |
| Fm | 2′-O-methylpseudouridine | Ms2t6a | N-((9-beta-D-ribofuranosyl-2-methylthiopurine-6- |
| | | | yl)carbamoyl)threonine |
| Gal q | Beta,D-galactosylqueosine | Mt6a | N-((9-beta-D-ribofuranosylpurine-6-yl)N-methyl- |
| | | | carbamoyl)threonine |
| Gm | 2′-O-methylguanosine | Mv | Uridine-5-oxyacetic acid methylester |
| I | Inosine | O5u | Uridine-5-oxyacetic acid (v) |
| I6a | N6-isopentenyladenosine | Osyw | Wybutoxosine |
| M1a | 1-methyladenosine | P | Pseudouridine |
| M1f | 1-methylpseudouridine | Q | Queosine |
| M1g | 1-methylguanosine | s2c | 2-thiocytidine |
| M1I | 1-methylinosine | s2t | 5-methyl-2-thiouridine |
| M22g | 2,2-dimethylguanosine | s2u | 2-thiouridine |
| M2a | 2-methyladenosine | s4u | 4-thiouridine |
| M2g | 2-methylguanosine | T | 5-methyluridine |
| M3c | 3-methylcytidine | t6a | N-((9-beta-D-ribofuranosylpurine-6- |
| | | | yl)carbamoyl)threonine |
| M5c | 5-methylcytidine | Tm | 2′-O-methyl-5-methyluridine |
| M6a | N6-methyladenosine | Um | 2′-O-methyluridine |
| M7g | 7-methylguanosine | Yw | Wybutosine |
| Mam5u | 5-methylaminomethyluridine | X | 3-(3-amino-3-carboxypropyl)uridine, (acp3)u |

A nucleobase may be comprised in a nucleoside or nucleotide, using any chemical or natural synthesis method described herein or known to one of ordinary skill in the art.

b. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Komberg and Baker, 1992).

c. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

d. Nucleic Acid Analogs

A tag or other nucleic acid used in the invention may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in U.S. Pat. No. 5,681,947 which describes oligonucleotides comprising purine derivatives that form triple helixes with and/or prevent expression of dsDNA; U.S. Pat. Nos. 5,652,099 and 5,763,167 which describe nucleic acids incorporating fluorescent analogs of nucleosides found in DNA or RNA, particularly for use as fluorescent nucleic acids probes; U.S. Pat. No. 5,614,617 which describes oligonucleotide analogs with substitutions on pyrimidine rings that possess enhanced nuclease stability; U.S. Pat. Nos. 5,670,663, 5,872,232 and 5,859,221 which describe oligonucleotide analogs with modified 5-carbon sugars (i.e., modified 2′-deoxyfuranosyl moieties) used in nucleic acid detection; U.S. Pat. No. 5,446,137 which describes oligonucleotides comprising at least one 5-carbon sugar moiety substituted at the 4′ position with a substituent other than hydrogen that can be used in hybridization assays; U.S. Pat. No. 5,886,165 which describes oligonucleotides with both deoxyribonucleotides with 3′-5′ internucleotide linkages and ribonucleotides with 2′-5′ internucleotide linkages; U.S. Pat. No. 5,714,606 which describes a modified internucleotide linkage wherein a 3′-position oxygen of the internucleotide linkage is replaced by a carbon to enhance the nuclease resistance of nucleic acids; U.S. Pat. No. 5,672,697 which describes oligonucleotides containing one or more 5′ methylene phosphonate internucleotide linkages that enhance nuclease resistance; U.S. Pat. Nos. 5,466,786 and 5,792,847 which describe the linkage of a substituent moeity which may comprise a drug or label to the 2′ carbon of an oligonucleotide to provide enhanced nuclease stability; U.S. Pat. No. 5,223,618 which describes oligonucleotide analogs with a 2 or 3 carbon backbone linkage attaching the 4′ position and 3′ position of adjacent 5-carbon sugar moiety to enhanced resistance to nucleases and hybridization to target RNA; U.S. Pat. No. 5,470,967 which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240 which describe oligonucleotides with three or four atom linker moeity replacing phosphodiester backbone moeity used for improved nuclease resistance; U.S. Pat. No. 5,214,136 which describes olignucleotides conjugated to anthraquinone at the 5′ terminus that possess enhanced hybridization to DNA or RNA; enhanced stability to nucleases; U.S. Pat. No. 5,700,922 which describes PNA-DNA-PNA chimeras wherein the DNA comprises 2′-deoxy-erythro-pentofuranosyl nucleotides for enhanced nuclease resistance and binding affinity; and U.S. Pat. No. 5,708,154 which describes RNA linked to a DNA to form a DNA-RNA hybrid.

e. Polyether and Peptide Nucleic Acids

In certain embodiments, it is contemplated that a tag or other nucleic acid comprising a derivative or analog of a nucleoside or nucleotide may be used in the methods and compositions of the invention. A non-limiting example is a “polyether nucleic acid”, described in U.S. Pat. No. 5,908,845, incorporated herein by reference. In a polyether nucleic acid, one or more nucleobases are linked to chiral carbon atoms in a polyether backbone.

Another non-limiting example is a “peptide nucleic acid”, also known as a “PNA”, “peptide-based nucleic acid analog” or “PENAM”, described in U.S. Pat. Nos. 5,786,461, 5,891,625, 5,773,571, 5,766,855, 5,736,336, 5,719,262, 5,714,331, 5,539,082, and WO 92/20702, each of which is incorporated herein by reference. Peptide nucleic acids generally have enhanced sequence specificity, binding properties, and resistance to enzymatic degradation in comparison to molecules such as DNA and RNA (Egholm et al., 1993; PCT/EP/01219). A peptide nucleic acid generally comprises one or more nucleotides or nucleosides that comprise a nucleobase moiety, a nucleobase linker moeity that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

In certain embodiments, a nucleic acid analogue such as a peptide nucleic acid may be used to inhibit nucleic acid amplification, such as in PCR, to reduce false positives and discriminate between single base mutants, as described in U.S. Pat. No. 5,891,625. Other modifications and uses of nucleic acid analogs are known in the art, and are encompassed by the invention. In a non-limiting example, U.S. Pat. No. 5,786,461 describes PNAs with amino acid side chains attached to the PNA backbone to enhance solubility of the molecule. Another example is described in U.S. Pat. Nos. 5,766,855, 5,719,262, 5,714,331 and 5,736,336, which describe PNAs comprising naturally and non-naturally occurring nucleobases and alkylamine side chains that provide improvements in sequence specificity, solubility and/or binding affinity relative to a naturally occurring nucleic acid.

f. Preparation of Nucleic Acids

A tag or other nucleic acid used in the invention may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotides are used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid includes one produced by enzymes in amplification reactions such as PCR (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989, incorporated herein by reference).

g. Nucleic Acid Purification

A tag or other nucleic acid used in the invention may be purified on polyacrylamide gels, cesium chloride centrifugation gradients, or by any other means known to one of ordinary skill in the art (see for example, Sambrook et al. 1989, incorporated herein by reference).

In particular embodiments, tags or other nucleic acid used in the invention may be isolated from at least one organelle, cell, tissue or organism. In certain embodiments, “isolated nucleic acid” refers to a nucleic acid that has been isolated free of, or is otherwise free of, the bulk of cellular components such as for example, macromolecules such as lipids or proteins, small biological molecules, and the like.

h. Nucleic Acid Complements

The present invention also encompasses a nucleic acid that is complementary to a specific nucleic acid sequence. A nucleic acid “complement(s)” or is “complementary” to another nucleic acid when it is capable of base-pairing with another nucleic acid according to the standard Watson-Crick, Hoogsteen or reverse Hoogsteen binding complementarily rules. As used herein “another nucleic acid” may refer to a separate molecule or a spatial separated sequence of the same molecule.

i. Hybridization

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suit a particular application.

B. Nucleic Acid Samples (Populations)

The invention can be applied to the comparative analysis of any nucleic acid population. The nucleic acids can be RNA, DNA, or both. The nucleic acids can be part of a collection of other molecules, including proteins, carbohydrates or small molecules. While the population can comprise even a single sequence, the method is best suited for nucleic acid samples that include hundreds or thousands of unique sequences.

The terms “target”, “target nucleic acid” and “target sequence” refer to one or more nucleic acids (e.g., DNA, RNA) of a specific sequence that are being characterized. Often, target nucleic acids comprise a sub-population of nucleic acids relative to all the nucleic acid sequences originally present in a nucleic acid sample.

1. Sources of Nucleic Acid Samples

Nucleic acid samples can be obtained from biological material, such as cells, tissues, organs or organisms. The invention is particularly relevant to total and polyA RNA preparations from tissues or cells. Similarly, the invention could be applied to cDNAs derived from cells or tissues. In other embodiments, multiple genomic DNA samples could be assessed using the methods of the present invention.

a. Cells and Tissues

A cell, or a tissue comprising cells, may be a source of nucleic acids for the present invention. In certain embodiments, cells or tissue may be part of or separated from an organism. In certain embodiments, a cell or tissue may comprise, but is not limited to, adipocytes, alveolar, ameloblasts, axon, basal cells, blood (e.g., lymphocytes), blood vessel, bone, bone marrow, brain, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, skin, small intestine, spleen; stem cells, stomach, testes, anthers, ascites, cobs, ears, flowers, husks, kernels, leaves, meristematic cells, pollen, root tips, roots, silk, stalks, and all cancers thereof.

b. Organisms

In certain embodiments, an organism may be a source of nucleic acids for the present invention. In certain embodiments, the organism may be, but is not limited to, a eubacteria, an archaea, a eukaryote or a virus (for example, webpage http://phylogeny.arizona.edu/tree/phylogeny.html).

i. Eubacteria

In certain embodiments, the organism is a eubacteria. In particular embodiments, the eubacteria may be, but is not limited to, an aquifecales; a thermotogales; a thermodesulfobacterium; a member of the thermus-deinococcus group; a chloroflecales; a cyanobacteria; a firmicutes; a member of the leptospirillum group; a synergistes; a member of the chlorobium-flavobacteria group; a member of the chlamydia-verrucomicrobia group, including but not limited to a verrucomicrobia or a chlamydia; a planctomycetales; a flexistipes; a member of the fibrobacter group; a spirochetes; a proteobacteria, including but not limited to an alpha proteobacteria, a beta proteobacteria, a delta & epsilon proteobacteria or a gamma proteobacteria. In certain aspects, an organelle derived from eubacteria are contemplated, including a mitochondria or a chloroplast.

ii. Archaea

In certain embodiments, the organism is an archaea (a.k.a. archaebacteria; e.g., a methanogens, a halophiles, a sulfolobus). In particular embodiments, the archaea may be, but is not limited to, a korarchaeota; a crenarchaeota, including but not limited to, a thermofilum, a pyrobaculum, a thermoproteus, a sulfolobus, a metallosphaera, an acidianus, a thermodiscus, a igneococcus, a thermosphaera, a desulfurococcus, a staphylothermus, a pyrolobus, a hyperthermus or a pyrodictium; or an euryarchaeota, including but not limited to a halobacteriales, methanomicrobiales, a methanobacteriales, a methanococcales, a methanopyrales, an archeoglobales, a thermoplasmales or a thermococcales.

iii. Eukaryotes

In certain embodiments, the organism is a eukaryote (e.g., a protist, a plant, a fungi, an animal). In particular embodiments, the eukaryote may be, but is not limited to, a microsporidia, a diplomonad, an oxymonad, a retortamonad, a parabasalid, a pelobiont, an entamoebae or a mitochondrial eukaryote (e.g., an animal, a plant, a fungi, a stramenopiles).

iv. Viruses

In certain embodiments the organism may be a virus. In particular aspects, the virus may be, but is not limited to, a DNA virus, including but not limited to a ssDNA virus or a dsDNA virus; a DNA RNA rev transcribing virus; a RNA virus, including but not limited to a dsRNA virus, including but not limited to a −ve stranded ssRNA or a +ve stranded ssRNA; or an unassigned virus.

c. Synthetic Samples

Nucleic acid samples comprising populations designed by the hand of man may also be generated and used as a standard against which another sample or subpopulation of target sequences could be compared. The synthetic population can be used to accurately quantify one or more targets from one or more sample(s) if the concentrations of the synthetic nucleic acids are known. For example, a synthetic sample may comprise a collection of nucleic acids (e.g., RNA, cDNA or genomic DNA) from many different tissues, cells (e.g., cell cultures), or other samples that could provide an average population against which a sample, or subpopulation of target sequences, could be compared. In another non-limiting example, the synthetic sample could comprise a collection of in vitro transcripts at known or unknown concentrations sharing a specific tag sequence so that they could be co-amplified with nucleic acids from another sample (e.g., RNA) to quantify a collection of targets. In another example, the synthetic sample could comprise a set of DNAs at known or unknown concentrations sharing a specific tag sequence that could be used to quantify a sample comprising a target DNA population.

d. Sample Mixtures

A sample mixture is a collection of two or more nucleic acid samples (e.g., RNA, cDNA or DNA). It is particularly preferred that the different nucleic acid samples (the “input samples”) that comprise the sample mixture are distinguishable. This is typically achieved by differentially tagging the targets of each input sample prior to mixing. In certain embodiments, a sample (e.g., an input sample) may comprise competitors. As used herein, a “competitor” is nucleic acid (e.g., RNA or DNA) that can be amplified by the same primers used to amplify one or more targets being assessed in a sample. In certain aspects, a competitor may be used to quantify one or more targets by comparing the abundance of the amplified and/or differentiated competitor(s) with the abundance of the amplified and/or differentiated target(s).

C. Functional Characteristics of Tags

The invention involves appending a tag to one or more target sequences, up to all nucleic acid sequences, comprised in a nucleic acid population. A tag is a common sequence shared by various nucleic acid sequences of a nucleic acid sample that allows nucleic acids of one population to be distinguished from another population. The term tag is also used to describe the RNA, DNA, or other nucleic acid molecule that is used to tag a nucleic acid in a sample. In preferred embodiments, a tag is an RNA, DNA, or other molecule that can be used as a template by a polymerase to generate a complementary strand.

A tag comprises at least two functional domains. The first, referred to as a “differentiation domain”, can be used to distinguish the nucleic acid target(s) derived from each sample (e.g., input samples in a sample mixture). The second functional element, referred to as an “amplification domain,” is used to amplify nucleic acid target sequences. Thus, in preferred embodiments, a tag comprises at least two functional domains, an amplification domain compatible with amplification and a differentiation domain that can be used to distinguish amplification products that derive from the sample(s) being assessed. Of course, a tag may comprise one or more additional sequences. Generally, additional sequences will possess functional properties, such as, for example, a property that facilitates analysis of amplified nucleic acids.

It is particularly preferred that the differentiation domain be between the amplification domain and the sequence of each target nucleic acid in the sample. In other words, it is particularly preferred that a differentiation domain is internal to the amplification domain.

The differentiation and amplification domain sequences can overlap, though it is particularly preferred that they are functionally distinct. This will help ensure that the amplified nucleic acids derived from a sample mixture can be distinguished in a way that is independent of their amplification.

1. Amplification Domains

In most embodiments, it is particularly preferred that a tag comprise at least one amplification domain. As used herein, an amplification domain will primarily be a sequence that can support the amplification of a nucleic acid that comprises such sequence. Use of nucleic acid sequences in amplification reactions are well known in the art, and non-limiting examples are described herein.

In particularly preferred embodiments, samples being assessed by the methods of the present invention are mixed with other samples to create a sample mixture. In embodiments wherein a sample mixture is assessed, the amplification domains of the tags used in the samples that were mixed will preferably be identical to facilitate equal co-amplification of the target sequences from the different input samples.

In certain embodiments, an amplification domain will comprise a sequence that can support primer binding and extension. Standard rules for primer design apply (Sambrook, 1994). In specific aspects, an amplification domain will preferably comprise a primer binding sites for PCR amplification. PCR™ does not require any specialized structure or sequence to sustain amplification; the PCR™ amplification primer typically contains only binding sequences. Parameters for primer design for PCR are well known in the art (see, e.g., Beasley et al., 1999).

Primer binding sites for other types of amplification methods might also be used as amplification domains. Often such primer binding regions share similar characteristics with PCR™ primer binding sites, however the primers used for other amplification methods typically possess sequences 5′ to the binding domain. For instance, primers for 3SR and NASBA contain an RNA polymerase promoter sequence 5′ to the priming site to support subsequent transcription. Because 3SR and NASBA are performed at relatively low temperature (37° C. to 42° C), the primer binding regions can have much lower melting temperatures than those used for PCR™.

2. Differentiation Domains

It is particularly preferred that a tag comprise at least one differentiation domain. A differentiation domain comprises a sequence that can be used to identify the sample from which a particular amplified nucleic acid derives. For example, a differentiation domain may comprise a different affinity sequence for removing one or more labeled nucleic acid(s) unique to each sample population (e.g., input sample populations in a sample mixture, a different primer binding domain for labeled DNA synthesis, a different transcription domain for labeled RNA synthesis, a size differentiation domain, an additional domain described herein or as would be known to one of skill in the art (e.g., a restriction enzyme site) or combinations thereof.

a. Primer Binding Domains

A differentiation domain may comprise a primer binding site (a “primer binding domain”). For example, a primer binding site may provide an annealing site for various types of primers that can be extended by a polymerase to generate a labeled nucleic acid (e.g., DNA). Binding sites for primers are well known in the art (Sambrook 1989).

b. Transcription Domains

In certain embodiments, a differentiation domain may comprise. a promoter sequence (a “transcription domain”) that binds an RNA polymerase to initiate transcription. In certain embodiments, the resulting differentiated RNA (e.g., a labeled RNA) is used for analysis. For example, an amplified population possessing promoter sequences can be transcribed in a reaction (e.g., an in vitro reaction) with one or more labeled nucleotides (radio- or non-isotopic-labeled NTPs) and an appropriate RNA polymerase to convert double-stranded nucleic acid amplification products into differentiated RNAs that can be used for comparative analysis.

c. Size Differentiation Domains

In certain embodiments, a differentiation domain may comprise a nucleic acid sequence of a different length than another differentiation domain. Such a nucleic acid sequence of a different length is known herein as a size differentiation domain.

d. Affinity Domains

In certain embodiments, a differentiation domain may provide an affinity site for hybridization or binding (an “affinity domain”) to a ligand comprising, but not limited to, a nucleic acid, protein or other molecule. For example, amplified nucleic acids or labeled nucleic acids generated from amplification products, can be divided into sample-specific fractions using affinity domains unique to each sample tag.

3. Additional Functional Domains

A tag may comprise one or more additional functional or structural sequences in addition to the primary amplification and primary differentiation domains, as described herein or as would be known to one of ordinary skill in the art. In certain embodiments, these domains may be partly or fully comprised within other domains, such as, for example an amplification domain or a differentiation domain. In other embodiments, these additional domains may be comprised in sequences that do not comprise the amplification domain or differentiation domain.

These additional domain(s) may be used to support additional molecular biological reactions, including but not limited to an amplification reaction, a differentiation reaction, a labeling reaction, a restriction digestion reaction, a cloning reaction, a hybridization reaction, sequencing reaction or a combination thereof. The addition of one or more additional domains will be particularly preferred in certain embodiments for manipulating the amplification products generated from targets in a sample mixture.

Additional sequences described herein are by no means intended as an exhaustive list of all of the potential functional domains that can be included to facilitate production, amplification, differentiation, comparison or analysis of nucleic acid targets in a sample. The list is merely intended to provide examples of some requirements and benefits of additional functional domains that can be incorporated into the nucleic acid tag.

a. Labeling Domains

A tag may comprise a sequence that is used in a labeling reaction (a “labeling domain”) to convert an amplified nucleic acid population into a labeled product population for subsequent analysis. A variety of sequences can be used to support the production of labeled products, and non-limiting examples are described herein. In specific embodiments, a labeling domain may be used for the synthesis of labeled DNA or labeled RNA. It is particularly preferred that the labeling domain be situated upstream of the differentiation domain so that the labeled nucleic acids include the differentiation domain sequence. In preferred aspects, the labeled nucleic acid products can then be distinguished using the unique differentiation domains prior to or during comparative analysis.

b. Primer Binding Sites for Sequence Analysis

A tag may comprise a primer binding site for a sequencing primer. For example, in certain preferred embodiments a primer binding site could be included in the tag sequence between the amplification and differentiation domains to facilitate sequence analysis of the differentiation domains of one or more amplified populations.

c. Restriction Enzyme Sites

A tag sequence may comprise one or more selected restriction enzyme sites, which may be used in various reactions, such as, for example, a cloning reaction.

In some embodiments, a restriction enzyme site may facilitate cloning of a nucleic acid comprising a tag. Methods of cloning are common in the art (Sambrook 1989). For example, cloning the amplified nucleic acid(s) resulting from competitive amplification will be particularly preferred to facilitate sequence analysis. Sequencing the amplification products can be used to determine the percentage of amplified nucleic acids bearing differentiation domains unique to each of the nucleic acid samples being compared.

In certain preferred aspects, a tag would comprise at least one restriction site on either side of a differentiation domain. In aspects wherein the restriction sites upstream and downstream of the differentiation domain were unique, then single differentiation domains could be directionally ligated into cloning vectors and subsequently sequenced.

In certain embodiments, restriction sites can be employed to facilitate concatenation for rapid sequence analysis as described in U.S. Pat. No. 5,866,330. For example, in aspects wherein the restriction sites were identical or otherwise able to be ligated, the differentiation domains could be ligated to one another to create extended chains of differentiation domains from amplified nucleic acids. In particular facets, the concatenated differentiation domains may be ligated into a cloning vector and subsequently sequenced to quantify the abundance of each differentiation domain in an amplified sample.

d. Secondary Amplification Domains

One or more amplification domains in addition to the primary amplification domain may be used for nested amplification(U.S. Pat. No. 5,340728). In general embodiments, nested amplification comprises sequential amplification reactions wherein a first amplification with a first set of one or more primers generates one or more primary amplified nucleic acids, and at least a second amplification of the one or more primary amplified nucleic acids with another set of primers comprising a primer that binds a sequence partly or fully internal to a primer of the first set, so that a nucleic acid segment of the one or more primary amplified nucleic acids is then amplified. In certain embodiments, nested amplification might be required for those targets that are present in only a few copies in a sample or where small amounts of a sample (e.g., a few mammalian cells) are available. The secondary amplification domain is typically between the primary amplification domain and the primary differentiation domain.

e. Secondary Differentiation Domains

One or more additional differentiation domains may be used in conjunction with the primary differentiation domain to further distinguish amplified nucleic acid targets. For example, if transcription is being used to differentiate targets amplified from their samples and only a few different polymerases are available for in vitro transcription, then only a few input samples can be assayed at a time. Incorporating a secondary differentiation domain between the amplification domain and the primary differentiation domain would allow additional samples to be mixed and assayed by the methods of the present invention. In one aspect, several samples could use tags with the same transcription promoter that comprises their primary differentiation domain so long as their secondary differentiation domains were unique. The primary amplification would use a single tag-specific primer for all samples. The amplified population could then be split and further amplified with primers specific to the secondary differentiation domains. Each of the resulting samples could then be used to generate differentiated populations for analysis using the different transcription promoters.

D. Methods for Appending Tags to Populations

A nucleic acid tag of the present invention may be added to or appended to a nucleic acid population. As would be appreciated by one of ordinary skill in the art, different methods of tag attachment or incorporation may be used depending on whether the nucleic acid population comprises DNA or RNA. Non-limiting examples of such methods that may be used are described herein, though other methods can be used as would be known by one of ordinary skill in the art.

1. Tagging RNA

The methods of the present invention are applicable to tagging eukaryotic RNA and/or prokaryotic RNA. In other aspects, the present invention may be applied to tag polyA selected or total RNA populations. As will be apparent to one of ordinary skill in the art in light of the disclosures herein, a tag may be appended to RNA populations in a variety of ways. Non-limiting examples of methods of tagging RNA are described below.

Once an RNA molecule is tagged, it can undergo further molecular biology reactions, including but not limited to, reverse transcription, amplification, transcription, prime extension, restriction digestion, sequencing, and/or hybridization. In preferred embodiments, amplification and differentiation can be accomplished using sequences present in the ligated tag. For example, a tagged mRNA population may be mixed with other tagged populations, converted to cDNA and the cDNA amplified with at least one primer specific to the tag and one or more primers specific to one or more target sequences in the samples. The amplified nucleic acids from the sample mixture may be differentiated using one of a variety of methods and assessed to compare the relative abundances of one or more RNA target(s) in the mRNA samples.

a. Ligation

In certain embodiments, a tag can be appended to the 3′ ends of RNAs by a ligase (e.g., an enzymatic protein, nucleic acid or chemical that induces ligation). For ligation, an excess of RNA or DNA polynucleotide tag possessing a 5′ phosphate can be added to a RNA population. Incubation of the mixture with a ligating agent (e.g., RNA ligase) generates RNAs with the tag ligated to the 3′ end of the RNAs.

In general embodiments, more efficient ligation may be achieved by adding bridging oligonucleotides to the ligation reaction. Hybridization of a bridge to both the sample nucleic acids (e.g., an RNA in the sample) and a tag will align the 3′ and 5′s ends of the two molecules, enhancing ligation efficiency. In a non-limiting example, a bridging oligonucleotide may comprise a sequence at its 3′ end that is complementary to the 3′ ends of RNAs in a sample and a sequence at its 5′ end that is complementary to the 5′ end of the tag.

b. Cap Dependent Ligation

In one embodiment, a cap dependent ligation may be used to selectively append tags to the 5′ ends of eukaryotic mRNAs. In general aspects, an RNA may be tagged by the combined enzymatic activities of a phosphatase (e.g., calf intestinal phosphatase), a pyrophosphatase (e.g., tobacco acid pyrophosphatase) that leaves a 5′ phosphate at the 5′ terminus of a capped message, and nucleic acid ligase (e.g., RNA ligase).

In a non-limiting example, a total RNA population is treated with calf intestinal phophatase (CIP) to dephosphorylate the RNA population. CIP is specific to RNAs with free terminal phosphates, therefore the 5′ phosphates of rRNAs, tRNAs, and partially degraded mRNAs are removed leaving these RNAs with 5′ hydroxyls. After the CIP is inactivated, the RNA preparation is treated with a phosphatase such as tobacco acid pyrophoshatase (TAP) to convert the 5′ cap structures of mRNAs to 5′ monophosphates. An excess of a DNA or RNA polynucleotide tag is added to the RNA population as well as a ligase that functions on RNA substrates. The tag should ligate exclusively to TAP modified RNAs possessing 5′ monophosphates as all of the non-capped RNAs possess 5′ hydroxyls following CIP treatment. The resulting tagged mRNA population can be used in subsequent reactions for comparative analysis.

c. Enzymatic Polymerization

In an additional embodiment, a tag is incorporated into an RNA population by enzymatic polymerization. An oligonucleotide tag comprising amplification and differentiation domains at its 5′ end and sequence complementary to the 3′ ends of RNA in a sample, and a 3′ nucleotide that cannot be extended by polymerization (see for example, U.S. Pat. No. 6,057,134), can be hybridized to the 3′ ends of an RNA population. An RNA or DNA polymerase with the ability to extend primer template junctions can be added to the mixture and allowed to extend the 3′ ends of the RNAs in the population, incorporating a sequence complementary to the hybridized oligonucleotide at the 3′ ends of the RNA in the sample. Because the oligonucleotide that serves as a template comprises a tag sequence, the polymerization reaction effectively tags the RNA sample population. The resulting nucleic acid can be mixed with other differentially tagged nucleic acids, reverse transcribed, amplified, and differentiated to compare targets in the RNA samples.

2. Tagging RNA Populations by Reverse Transcription

In a preferred embodiment, tag sequences may be appended to sample nucleic acids by reverse transcription. For example, tagged cDNA populations can be conveniently generated by priming reverse transcription with oligonucleotides comprising a tag sequence at its 5′ end and sequence complementary to RNAs in a sample at its 3′ end. Hybridization of the primer to one or more targets in an RNA sample and subsequent reverse transcription yields cDNA with tag sequences at its 5′end.

For example, most eukaryotic mRNAs possess a polyA tail that can be tagged with a primer that has a polyT or polyU at or near its 3′ end and an amplification and a differentiation domain at its 5′ end. The polyA specific tag primer can be extended from the polyA tail of the mRNAs. The resulting cDNAs possess the tag sequences at or near their 5′ ends that may be used in subsequent amplification and differentiation reactions.

3. CAPswitch™

A method for tagging mRNAs by Cap-induced primer extension is described in U.S. Pat. No. 5,962,271. The technology, referred to as CAPswitch™, uses a unique CAPswitch oligonucleotide in the first strand cDNA synthesis reaction. When reverse transcriptase stops at the 5′ end of an mRNA template in the course of first strand cDNA synthesis, it switches to a CAPswitch oligonucleotide and continues DNA synthesis to the end of a CAPswitch oligonucleotide. The resulting cDNA has at its 3′ end a sequence that is complementary to the CAPswitch oligonucleotide sequence. The CAPswitch technology may be used to tag one or more RNA populations by using one or more CAPswitch oligonucleotides comprising differentiation and amplification domains.

4. Tagging DNA

DNA (e.g., genomic DNA and cDNA) can be tagged by various methods, including primer extension or ligation.

a. Single Stranded DNA

In one embodiment, a single-stranded DNA (e.g., cDNA) population may be diluted in a buffer appropriate for hybridization and polymerization, and hybridized to one or more tags comprising specific or random sequences at their 3′ ends and amplification and differentiation domain at their 5′ ends. Addition of a DNA polymerase such as, for example, the klenow fragment of DNA polymerase I or Taq DNA polymerase, will extend a tag to create a tagged population of DNA segments.

In aspects where the DNA is double stranded (e.g., genomic DNA), it may be denatured prior to tagging by any of a variety of methods known in the art, including, for example, heating to 95° C. in a solution of 0.2 M NaOH. In certain aspects, the denatured DNA may be removed or purified from. the denaturing reagents by methods well known to those of skill in the art, such as, for example, ethanol precipitation. The denatured DNA may then be tagged using primer extensions as described herein or as would be known to one of ordinary skill in the art.

b. Double Stranded DNA

In certain embodiments, double-stranded DNA may be tagged by ligation. For example, a double-stranded DNA can be digested with a restriction enzyme, and one or more double stranded tags comprising a compatible restriction fragment cut site may be ligated to the digested DNA.

A disadvantage of appending double-stranded tags to double-stranded nucleic acids (e.g., DNA) is that primers specific to the amplification domain of the tag can bind and be extended from target and non-target molecules alike. Using restriction digestion and double-stranded tag ligation may create far greater background than the other methods described for tagging a nucleic acid target and is therefore a less preferred method for tagging populations. This is in contrast to other tagging methods described herein, whereby single-stranded tags are appended to single-stranded nucleic acids from the sample. In these embodiments, the amplification domain of the tag sequence only becomes a primer binding site when the target specific primer is extended during the amplification phase.

E. Amplification

After differentially tagged samples are mixed, the sample mixture may be amplified to generate an amplified population comprising a set of distinct amplified nucleic acids.

For amplification reactions, it is preferred to remove any unincorporated tags prior to amplification to keep the tag and amplification primer from competing for templates during amplification. A primer can be removed from the sample using, for example, size exclusion chromatography (Sambrook 1989). In a preferred embodiment, supports with a pore size large enough to allow the tags to enter while excluding the larger nucleic acids provides an easy way to generate primer-free nucleic acids. In other embodiments, the free tags can be removed from a nucleic acid population by differential precipitation. For example, LiCl and ethanol are both known to preferentially precipitate larger DNA, therefore, as would be known to one of ordinary skill in the art, appropriate conditions may be developed to separate DNA from the oligonucleotide tags prior to amplification.

1. General Amplification Techniques

A number of template dependent processes are available to amplify sequences present in a given sample. A non-limiting example is the polymerase chain reaction (referred to as PCR) which is described in detail in U.S. Pat. Nos. 4,683,195, 4,683,202 and 4,800,159, and in Innis et al., 1988, each of which is incorporated herein by reference in their entirety. Other non-limiting methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

In another embodiment, a reverse transcriptase PCR amplification procedure may be performed to amplify mRNA populations. Methods of reverse transcribing RNA into cDNA are well known (see Sambrook, 1989). Alternative methods for reverse transcription utilize thermostable DNA polymerases. These methods are described in WO 90/07641. Additionally, representative methods of RT-PCR are described in U.S. Pat. No. 5,882,864.

Other non-limiting nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in. their entirety). European Application No. 329 822 discloses a nucleic acid amplification process involving cyclically synthesizing single-stranded RNA (“ssRNA”), ssDNA, and double-stranded DNA (dsDNA), which may be used in accordance with the present invention.

a. Nucleic Acid Sequence Based Amplification

Nucleic Acid Sequence Based Amplification (NASBA) (Guatelli, 1990; Compton, 1991) makes use of three enzymes, avian myeloblastosis virus reverse transcriptase (AMV-RT), E. coli RNase H, and T7 RNA polymerase to induce repeated cycles of reverse transcription and RNA transcription. The NASBA reaction begins with the priming of first strand cDNA synthesis with a gene specific oligonucleotide (primer 1) comprising a T7 RNA polymerase promoter. RNase H digests the RNA in the resulting DNA:RNA duplex providing access of an upstream target specific primer(s) (primer 2) to the cDNA copy of the specific RNA target(s). AMV-RT extends the second primer, yielding a double stranded cDNA segment (ds DNA) with a T7 polymerase promoter at one end. This cDNA serves as a template for T7 RNA polymerase that will synthesize many copies of RNA in the first phase of the cyclical NASBA reaction. The RNA then serve as templates for a second round of reverse transcription with the second gene specific primer, ultimately producing more DNA templates that support additional transcription.

In certain embodiments, NASBA could be adapted to the present invention to provide competitive amplification of target sequences. For example, the amplification domain of the tag sequence would comprise a promoter for an RNA polymerase and a primer binding site downstream of the promoter. A nucleic acid primer would initiate amplification by driving complementary strand synthesis from a target sequence. If the sample mixture comprised DNA, then the resulting double-stranded nucleic acid would be a template for transcription. If the sample mixture comprised RNA, then a primer specific to the amplification domains of. the samples would bind the cDNA of the first strand reaction and prime synthesis of a double-stranded template. In either case, the double stranded DNA would be trancribed by the action of the RNA polymerase and the resulting transcripts would be reverse transcribed and further converted to transcription templates by the actions of the primers and enzymes in the NASBA reaction. The amplified nucleic acids (e.g., RNA or cDNA) could be quantified using the unique differentiation domains of the appended tags. The ratio of amplified nucleic acids with each different differentiation domain would reflect the relative abundance of the target sequence in the samples.

b. Strand Displacement Amplification

Strand Displacement Amplification (SDA) is an isothermal amplification scheme that consists of five steps: binding of amplification primers to a target sequence, extension of the primers by an exonuclease deficient polymerase incorporating an alpha-thio deoxynucleoside triphosphate, nicking of the hemiphosphorothioate double stranded nucleic acid at a restriction site, dissociation of the restriction enzyme from the nick site, and extension from the 3′ end of the nick by an exonuclease deficient polymerase with displacement of the downstream non-template strand. Nicking, polymerization and displacement occur concurrently and continuously at a constant temperature because extension from the nick regenerates another hemiphosphorothioate restriction site. In embodiments wherein primers to both strands of a double stranded target sequence are used, amplification is exponential, as the sense and antisense strands serve as templates for the opposite primer in subsequent rounds of amplification.

In some embodiments, SDA may be adapted to the present invention to provide competitive amplification of target sequences. For example, the amplification domain of the tag sequence would comprise a primer binding site and an appropriate restriction enzyme site. A sample mixture may be added to an SDA reaction with tag and target specific primers with associated restriction sites compatible with SDA. The primers could be extended and the extended nucleic acids could be digested by restriction enzymes specific to the restriction sites in the tag and target primers. The digested nucleic acids would serve as templates for subsequent cycles of primer extension and restriction digestion. The final amplified nucleic acids would be assessed to determine the relative abundance of amplified nucleic acids possessing each of the sample-specific differentiation domains.

c. Transcription

DNA molecules with promoters can be templates for any one of a number of RNA polymerases (Sambrook 1989). An efficient in vitro transcription reaction can convert a single DNA template into hundreds and even thousands of RNA transcripts. While this level of amplification is orders of magnitude less than what is achieved by PCR, NASBA, and SDA, it could be sufficient for some embodiments of the present invention.

In certain embodiments, to use transcription as an amplification step in the present invention, the amplification domains of the tags would comprise identical transcription promoters. Differentially tagged nucleic acid samples could be added to primer extension reactions to make double-stranded RNA from targets in the sample mixture. The double-stranded DNA could be added to an in vitro transcription reaction with a polymerase appropriate to the promoter sequence of the tag amplification domain. Following transcription, the differentiation domains of the RNA population may be used to determine the relative abundance of target RNA derived from each of the nucleic acid samples.

d. Rolling Circle Amplification

Rolling circle amplification has been used to detect target nucleic acids (Lizardi, 1998; Zhang, 1998). This amplification reaction uses a circular nucleic acid template. Linear templates are typically circularized by hybridizing the 5′ and 3′ ends of the template to a single nucleic acid molecule that brings the terminal template nucleotides into close proximity. A ligase is added to circularize the template. A primer complementary to the circular RNA or DNA then hybridizes and initiates primer extension. Using a polymerase with strand-displacing activity allows the extended nucleic acid to be infinitely long. To achieve exponential amplification, a primer specific to the displaced ssDNA nucleic acid is added to the reaction. Multiple copies of the second primer can hybridize along the length of the Rolling Circle product nucleic acid. Extension and strand displacement at the multiple sites produces complementary molecules. Priming off of these nucleic acids by the first primer contributes to the accumulation of target dependent nucleic acid synthesis.

In some embodiments, Rolling Circle Amplification may be adapted to the present invention to provide competitive amplification of targets in a sample mixture. For example, RNA populations could be reverse transcribed using oligonucleotide tags. For each target cDNA being assayed, a polynucleotide would be synthesized that possessed sequence at its 3′ end that is complementary to the 5′ end of the tag sequence and at its 5′ end sequence complementary to the 3′ end of the target cDNA. Following hybridization to the targets in the sample mixture, the target cDNA would be ligated to circularize the template. A primer specific to the amplification domain of the appended tags would be added to initiate rolling circle amplification. The differentiation domains of the amplified nucleic acids may be used to determine the relative number of amplification products derived from each input sample in order to determine the abundance of the target in each of the input samples.

F. Differentiation

Differentiation is any of a variety of methods that distinguish from which sample a particular amplified nucleic acid derives. In general embodiments, the differentiation domains of amplified nucleic acids are used to identify sequences that derive from a sample. In preferred embodiments of the invention, a differentiation reaction is accomplished using the differentiation domain of appended tags. For example, following amplification, the differentiation domain is used to generate a differentiated nucleic acid population that can be used for analysis. In another non-limiting example, a differentiation domain is used to differentiate amplified populations without the creation of a distinct differentiated nucleic acid population.

1. Differential Labeling by Primer Extension

In certain embodiments, a differentiation domain comprises a differentiation primer binding site internal to the amplification domain. The primer binding site is functionally distinct for each sample population. In certain facets, a differentiation primer may be hybridized to a binding site and extended by a DNA polymerase (e.g., klenow fragment of DNA polymerase I or Taq DNA polymerase) to produce a differentiated nucleic acid from the amplified population. In a preferred facet, the differentiated nucleic acid comprises a labeled nucleic acid.

As used herein, a “labeled product” or a “labeled nucleic acid” is a nucleic acid that includes a detectable molecule or moiety (a “labeling agent”). Labeling agents include non-isotopic reagents, isotopic reagents or combinations thereof. Non-isotopic compounds used for labeling are typically an affinity ligand such as, for example, a biotin, a digoxigenin, or a DNP or a fluorescent dye such as Cy3 or Cy5 that are attached covalently to a primer, one or more dNTPs being incorporated, or both. Alternatively, one or more radiolabeled atoms (e.g., ³²P, ³³P, or ³⁵S) may be incorporated into the primer, dNTPs, or both. Of course, other labeling agents that would be known to those of skill in the art in light of the disclosures herein may be used.

In some aspects, a differentiation primer can be hybridized to an amplified population and extended using labeled nucleotides. In embodiments wherein labeled nucleotides are being incorporated, it is preferred to keep amplification primers from being extended during the labeling reaction. Because the primers used to amplify can hybridize equally well to all of the sample populations, the labeled nucleic acids resulting from the extension of any non-differentiation primers would be as likely to derive from an unintended sample as an intended sample. The labeled nucleic acid would therefore not be specific to a single input sample making the labeled nucleic acids incompatible with comparative analysis.

Thus, in particularly preferred aspects, the non-differentiation primers are removed from the amplified population (e.g., a sample mixture) prior to initiating a differentiation reaction. A primer can be removed using techniques that would be known to those of skill in the art, such as for example, size exclusion chromatography or precipitation of nucleic acids using conditions that keep primers in solution (Sambrook, 1996). For example, a nucleic acid population can be added to a size exclusion column and centrifuged. The amplified population collects in the filtrate, free of the column-bound amplification primers.

In certain embodiments, the differentiation primers are labeled and labeled nucleotides are not incorporated during primer extension. A benefit of using labeled differentiation primers in reactions without labeled nucleotides is that a single primer extension reaction can be used to differentially label amplification products from each (e.g., all) of the various samples comprising a sample mixture. For example, if the differentiation primer used to label amplification products derived from one sample has Cy3 and the differentiation primer for amplification products derived from the second sample has Cy5, then the two primers could be hybridized to amplification products and extended by the action of a DNA polymerase. Targets derived from one sample would be labeled exclusively with Cy3 while targets from the second sample would be labeled with Cy5. Target detection would be performed in a way that the signals from Cy3 and Cy5 could be distinguished, providing a measure of the relative abundance of each of the targets from the two samples (Chee 1996).

2. Differential Labeling by In Vitro Transcription

In embodiments wherein the tags of the amplified DNA population include a transcription promoter, a transcription reaction with one or more labeled nucleotides (e.g., isotopic- or non-isotopic-labeled NTPs) and an appropriate RNA polymerase can be used to convert double-stranded templates into differentiated RNAs that can be used for comparative analysis. For example, where the differentiation domains of different samples possess unique promoters, the amplified products generated from target(s) in a sample mixture can be split into multiple transcription reactions specific to each transcription promoter. Transcription reactions incorporating one or more labeled NTPs create labeled RNAs specific to each input sample. The labeled RNAs can be used to compare the abundance of targets in each of the nucleic acid samples.

RNA polymerases are well known to those of ordinary skill in the art. For example, several phage RNA polymerases have been isolated and characterized (Sambrook 1996). Additional RNA polymerases may be isolated from nature or by a mutation/selection screen using an existing polymerase (Ikeda 1993). Any such polymerases or promoters are contemplated for use in the present invention.

3. Differentiation by Affinity Purification

A differentiation domain of a tag may comprise a sequence with an affinity for a specific nucleic acid, protein, or other binding ligand. A binding ligand may comprise, but is not limited to, an oligonucleotide complementary to a differentiation domain, a nucleic acid binding protein (e.g., a transcription factor) that binds to a specific DNA or RNA sequence, a small molecule that intercalates into a given RNA or DNA sequence or combinations thereof. A binding ligand may either be bound to a solid support (e.g., a single bead or a membrane in the form of an array) or otherwise readily removed or separated from a solution.

In certain embodiments, the methods of the present invention may comprise a labeling step to provide labeled nucleic acids from the amplified target nucleic acids synthesized from a sample mixture. In specific aspects, the labeled nucleic acids would be applied to solutions or solid supports possessing ligands specific to the differentiation domains of the samples. The specifically isolated, labeled nucleic acids could then be compared to the unbound or differentially bound nucleic acids from other samples to compare the abundance of targets in the samples being compared.

4. Differentiation by Sequence Analysis

Because the sequences of the differentiation domains are unique, methods for sequence analysis that are known in the art could be used to assess the population of amplified nucleic acids to determine the relative abundance of targets present in each sample. In embodiments wherein only a few samples are mixed, amplified, and characterized, the population of amplified nucleic acids could be sequenced directly. The relative abundance of each differentiation domain could be determined by measuring the relative intensity of bands at each sequencing position. Provided that the positions being quantified were unique for each differentiation domain, the band intensity for each different nucleotide in the peak would correspond to the relative abundance of that amplified target nucleic acid in the sample.

Another method for quantifying amplified nucleic acids by sequence analysis involves cloning and sequencing. The amplified nucleic acids would be ligated into cloning vectors, the resulting plasmids would be used to transform a suitable host such as E. coli, the transformed sample would be used to isolate clones, and the clones would be sequenced using methods common in the art (Sambrook 1989). The number of clones possessing each differentiation domain would be tallied to reveal the make-up of the amplified population.

In another embodiment, cloning of amplified nucleic acids may be accomplished without the use of restriction digestion. For example, U.S. Pat. No. 5,487,993 takes advantage of the activity of many thermostable polymerases whereby a non-templated dATP is attached to the 3′ ends of PCR amplified nucleic acids. The PCR amplified nucleic acids can be readily ligated into linearized vectors possessing single T overhangs at their 3′ ends without restriction digestion of the amplified nucleic acids. It is contemplated that this method could be incorporated into the present invention by providing a rapid method to clone the amplified nucleic acids. The cloned amplified nucleic acids could be sequenced using any of the methods common in the art.

U.S. Pat. No. 5,695,937 describes another technique that could facilitate the sequencing of amplified nucleic acids generated in the practice of the present invention. Serial analysis of gene expression (SAGE) is a method that allows for the rapid quantitative analysis of independent nucleic acids. The method involves digesting DNA populations with restriction enzymes that generate short, double-stranded oligomers. The oligomers are ligated together, cloned, and sequenced. A single sequencing run can provide the identity of 20 to 50 oligomers, for example. Because each oligomer represents a unique member of the sample's DNA population, the identities of members of a nucleic acid sample can be determined. Several sequencing runs can provide statistically significant quantitative data on the relative abundance of the targets that comprise a sample.

SAGE could facilitate the quantitative analysis of amplified populations generated by protocols incorporating the methods of the present invention. To use SAGE, tag sequences would preferably comprise appropriate restriction sites upstream and/or downstream of the differentiation domains. The amplified population would be digested with restriction enzymes, the differentiation domains would be concatenated and cloned, and the clones would be sequenced. The sequenced differentiation domains would be quantified to reveal the relative abundance of target sequences in each of the samples.

5. Differentiation by Hybridization in Solution

In other embodiments, the amplified population can be analyzed in solution. For example, U.S. Pat. Nos. 5,210,015 and 6,037,130 describe techniques that detect amplified nucleic acids possessing specific sequences. Either of these two methods could be used to quantify amplified targets generated with the methods of the present invention. In one embodiment, oligonucleotides (e.g., labeled probes) specific to each of the differentiation domains present in the tag of a each of the samples being mixed and co-amplified could be hybridized to an amplified population. The amount of signal from each different oligonucleotide would reveal the relative abundance of each sample-specific differentiation domain. In embodiments wherein the differentiation domain-specific oligonucleotides are labeled with the same or indistinguishable detectable moiety, the differentiation domains would need to be quantified separately with each of the different oligonucleotides. Alternatively, oligonucleotides labeled with distinguishable moieties could be used in a single detection reaction to quantify multiple differentiation domains in products of amplification. The latter method would be preferred as it facilitates rapid analysis.

6. Differentiation by Electrophoretic Mobility/Size

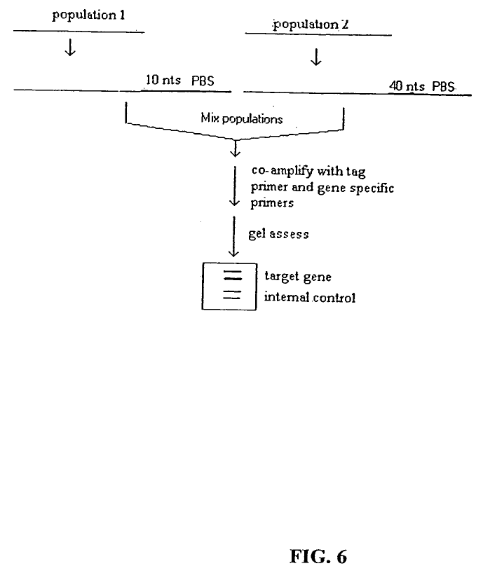

Gel and capillary electrophoresis can be used to assay co-amplified nucleic acids provided the differentiation domains of different sample populations are different sizes. For example, multiple samples with identical amplification domains but distinct sized differentiation domains can be mixed (FIG. 6). The sample mixture can be amplified using a primer specific to the amplification domain of the tag and a primer specific to the desired target. The products of amplification can be fractionated by size to reveal the samples from which they derive. The abundance of the discreet sized amplified nucleic acids would reveal the relative abundance of the targets in the samples.

In addition to capillary and gel electrophoresis, separation of nucleic acids may be conducted by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

G. Identifying a Tag

Because unique tags are used for different sample populations, it will be very important that the unique tags not contribute to amplification or differentiation biases (e.g., differences in amplification or differentiation efficiencies). New tag sequences should be tested to ensure that they function equivalently. The most powerful experiment contemplated for such a comparative test involves splitting a single sample into separate tagging reactions incorporating the different tags. After tagging, the samples are mixed, amplified, and differentiated. The differentiated nucleic acids are assessed using the method that is to be applied for analysis. For example, if the tags are to be used for differential display, then the differentiated nucleic acids are assessed by electrophoresis on adjacent lanes of an acrylamide gel (Sambrook, 1989). If the number of bands, the migration of the bands, or the intensity of the bands varies in the analysis of the differentiated nucleic acid population, then the tags are not functioning equivalently. Alternatively, if the tags are to be used for array analysis, then the labeled nucleic acids of the differentiation reactions should be hybridized to arrays. Once again, if the tags are functioning equivalently, then the probe spots should be identical as they were generated from the same sample population. If signal variation occurs in whatever analysis is being used, then the tags are biasing the analysis and should be redesigned.

Identifying differentiation domains that function equally well and that do not affect amplification efficiency is relatively straightforward where primer extension, affinity purification or digestion is being used for differentiation. In these cases, altering the identity of just a few nucleotides can provide effective differentiation (e.g., labeling specificity); rarely does altering a few bases within the differentiation domain affect amplification efficiency. In addition, because both methods use the same enzyme (i.e., a single DNA polymerase) for generating labeled nucleic acids from each of the unique tags, polymerization biases should not introduce variability.

However, where in vitro transcription is used for differentiation, amplification and/or differentiation bias is far more likely to occur. Promoters for the well-characterized phage RNA polymerases are similar in base content, but they stretch over 15-20 nucleotides creating a relatively large, unique sequence domain within the amplified nucleic acids. In addition, different RNA polymerases are used for each different differentiation reaction. Because the different polymerases are likely to possess sequence biases that affect transcription efficiency, the differentiated nucleic acids might not reflect the input samples. This has not affected the method of the present invention in the examples conducted and described herein. However, it is possible that this may affect certain embodiments. To overcome these potential problems, mutants of a single RNA polymerase that do not affect enzymatic activity but do alter promoter specificity may be used in the methods of the present invention may be designed (Ikeda 1993). This methodology may allow the creation of promoter sequences and mutant polymerases that provide equal amplification and differentiation efficiency to be used to distinguish differentially tagged amplified nucleic acids.

### H. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Population Tagging

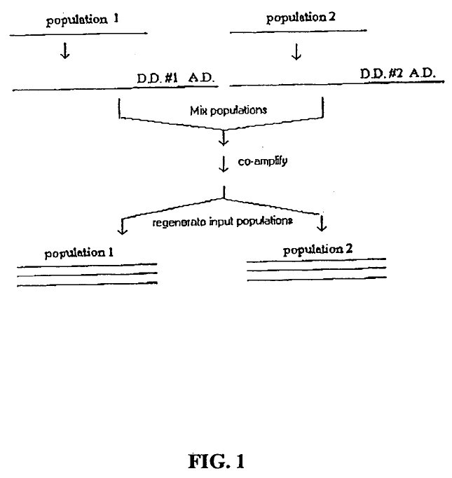

FIG. 1 depicts a general scheme of the aspects of the invention that allow for comparison of at least a first nucleic acid target within two or more populations. Thick lines represent tag sequences and thin lines represent sequences of the RNA and DNA populations comprising one or more target nucleic acids. A first nucleic acid tag comprising an amplification domain (A.D.) and a differentiation domain (D.D.#1) is appended to a first nucleic acid target of a first nucleic acid population. A second nucleic acid tag comprising an amplification domain and a different differentiation domain (D.D.#2) is appended to the first nucleic acid target of at least a second input nucleic acid population.

The first nucleic acid target can be one of a plurality of nucleic acid targets, and the first and second populations can be part of a plurality of populations being analyzed.

The tagged target(s) in the sample mixture are co-amplified, producing at least a first amplified nucleic acid comprising at least a differentiation domain of the first nucleic acid tag and a nucleic acid segment of the target(s), and at least a second amplified nucleic acid comprising at least a differentiation domain of the second nucleic acid tag and a nucleic acid segment of the target(s). Amplification of the target(s) of the two populations is achieved using a primer or polymerase specific to the A.D. in all tags.

The amplified nucleic acids are differentiated using the unique differentiation domains (D.D.#1 and D.D.#2) and the differentiated nucleic acids derived from population #1 and population #2 are compared to determine the abundance (i.e., concentration) of the first nucleic acid target in the first sample relative to the abundance of the first nucleic acid target in the second sample.

### Example 2

### Differential Labeling of Amplified Samples by Primer Extension

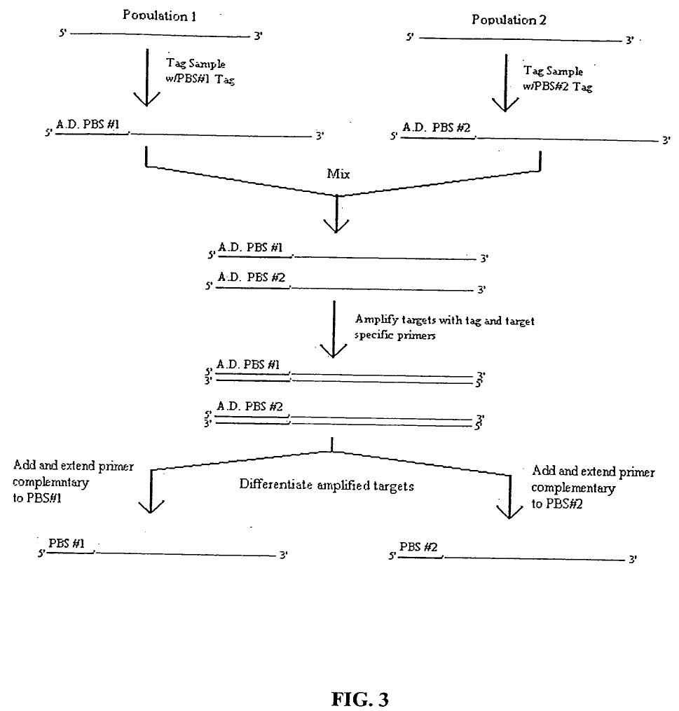

FIG. 3 depicts one of the most common embodiments of the invention, in which the same nucleic acid target is comprised within two or more populations. The thick lines in FIG. 3 represent the tag sequences. The thin lines represent the sequences of the RNA and/or DNA populations in which one or more nucleic acid targets are comprised. A first nucleic acid tag comprising a differentiation domain having a first primer binding domain (PBS#1) is appended to the nucleic acid target of a first nucleic acid population. A second nucleic acid tag comprising a differentiation domain having a second primer binding domain (PBS#2) is appended to the nucleic acid target of a second nucleic acid population. The differentiation domain of the second nucleic acid tag is different than the differentiation domain of the first nucleic acid tag.

FIG. 3 shows only one target and only two populations. However, the nucleic acid target may be one of a plurality of nucleic acid targets comprised in the population. Further, the first and second populations may be two of a plurality of populations being analyzed. In the protocol, at least two nucleic acid samples are mixed to produce a sample mixture.

The tagged target(s) in the sample mixture are amplified using tag and target-specific primers. The amplified nucleic acid targets are differentiated using labeling primer extension reactions using primers specific to the differentiation domains of the different samples. The differentiated nucleic acids are compared to determine the abundance (i.e., concentration) of the first nucleic acid target in the first population relative to the abundance of the first nucleic acid target in the second population.

### Example 3

### Differential Labeling of Amplified Samples by Transcription

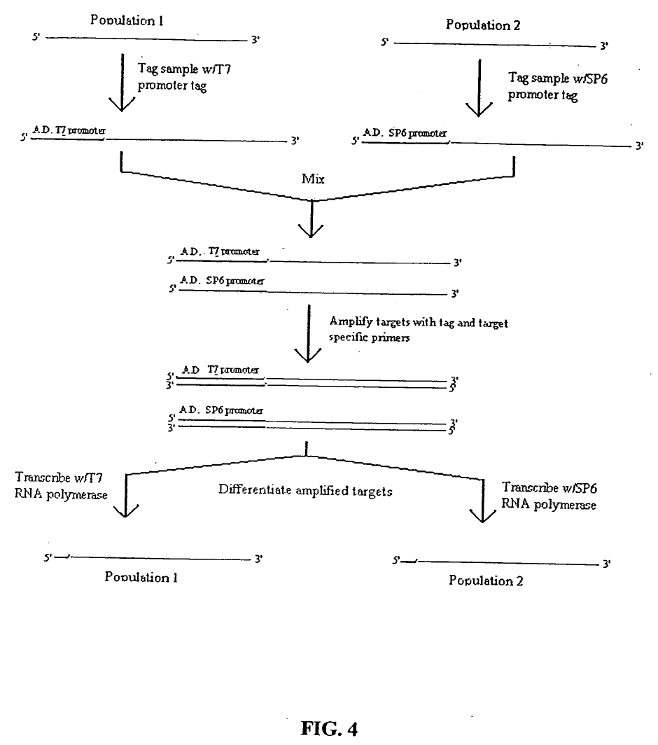

FIG. 4 depicts the application of the invention to compare at least a first nucleic acid target within two or more populations.

In this application, a nucleic acid tag comprising a differentiation domain that is a first transcription domain (i.e., a T7 promoter) is appended to a first nucleic acid target of a first nucleic acid population. A second nucleic acid tag comprising a differentiation domain that is a second transcription domain (i.e., a SP6 promoter) is appended to the first nucleic acid target of a second nucleic acid population. The transcription domain forming the differentiation domain of the second nucleic acid tag is specific for a different polymerase than that in the differentiation domain of the first nucleic acid tag. Any form of promoter and polymerase combination may be used, and the T7 and SP6 promoters, while very useful in the invention, are not limiting.

Of course, the first nucleic acid target can be only one of a plurality of nucleic acid targets and the first and second populations may be only two members of a plurality of populations being analyzed. However, for the sake of clarity, only one target and two populations are shown in this figure.

In FIG. 4, the thick lines represent the tag sequences. The thin lines represent the sequences of the RNA and/or DNA populations in which the one or more nucleic acid targets are comprised.

In the practice of the embodiment of the invention as shown in FIG. 4, two or more nucleic acid populations are mixed to produce a sample mixture. The tagged target(s) in the sample mixture are amplified using tag and target specific primers. The collection of amplification products can then be differentiated by transcription with RNA polymerases specific to the transcription promoters comprising the differentiation domains of the two samples.

### Example 4

### Differential Labeling of Amplified Samples by Affinity Isolation

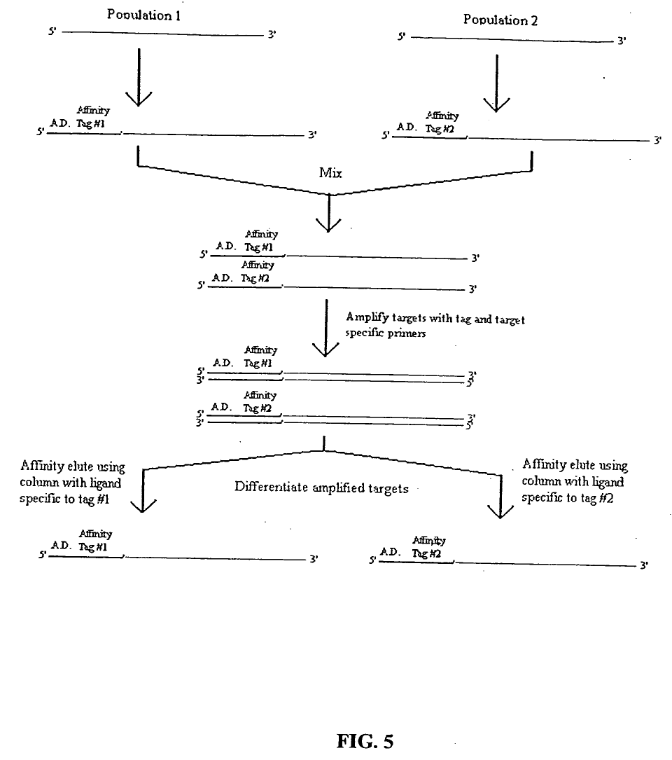

Multiple nucleic acid samples can be differentiated using sequences with affinities for different ligands (proteins, oligonucleotides, or small molecules). This is shown in FIG. 5, where target sequences are represented by thin lines, and appended tag sequences are drawn as thick lines. Differentiation domains with affinities for different ligands are labeled as Affinity Tag #1 and Affinity Tag #2. tags with unique affinity domains are used to differentially tag multiple RNA or DNA samples.

The differentially tagged cDNAs are mixed and target(s) present in the sample mixture are amplified using one primer specific to the amplification domain of the tag and one or more primers specific to nucleic acid targets. A labeled nucleotide or primer can be incorporated during the amplification reaction or the amplification products can be used in a subsequent labeling reaction (for instance, a transcription reaction) provided that an appropriate labeling domain is present in the tag sequences. The labeled nucleic acids derived from each sample are distinguished using ligands specific to each affinity domain appended to the various tags. For instance, oligonucleotides specific to each affinity domain could be attached to different beads. Each of the sample specific beads could be incubated with the labeled nucleic acids, then removed to provide labeled targets specific to each sample. The labeled nucleic acids could then be applied to any of a variety of techniques to assess the relative abundance of targets in each of the nucleic acid samples. For instance, each of the labeled nucleic acid fractions could be applied to an array to distinguish the signal from each of the targets derived from each sample. The array data generated from one sample can be compared to another to reveal the relative abundance of targets in each sample.

### Example 5

### Quantitative Analysis Using Size Differentiation Domains

There is great interest in identifying differentially expressed genes and a number of techniques have been developed to facilitate the search (SAGE, differential display, array analysis, and other techniques known to those of skill). Confirming differential expression once the primary screen is complete tends to be very tedious. Northern blotting requires that probes be made for each gene target and that 2-3 days be spent hybridizing, washing, and exposing blots for each target. RPAs share similar problems. Relative RT-PCR tends to be difficult to set up and only moderately quantitative.

One application of the invention uses differentiation domains that are different sizes. Following amplification of target(s) in a sample mixture, the amplification products are distinguished by size. The inventors refer to the method as comparative RT-PCR. Comparative RT-PCR is ideally suited for confirming and quantifying targets that appear to be differentially expressed.

Comparative RT-PCR comprises reverse transcribing different mRNA populations using anchored oligodT primers with identical primer binding sites at their 5′ ends (amplification domains) and different length polynucleotide linkers between the primer binding site and oligodT that function as differentiation domains. Two or more differentially tagged cDNA populations are mixed and amplified by PCR using one primer specific to the tags and one or more primer(s) specific to a gene(s) of interest. The resulting amplified nucleic acids are differentiated by fractionation using gel electrophoresis. Because the appended tags are different sizes for the different populations, the amplified nucleic acids that result from different populations migrate differently in the gel. These differentiated nucleic acids are then quantified to provide the relative expression of the target(s) in each of the populations. A specific example of this protocol is shown in FIG. 6.

In FIG. 6, a first nucleic acid tag comprising an amplification domain (e.g., a primer binding domain) and a differentiation domain comprising a first size differentiation domain (i.e., 10 nucleotides in length) is appended to a first nucleic acid target of a first nucleic acid population. A second nucleic acid tag comprising an amplification domain (e.g., a primer binding domain) and a differentiation domain wherein the differentiation domain comprises a second size differentiation domain (i.e., 40 nucleotides in length) is appended to the first nucleic acid target of a second nucleic acid population. While the sizes of the differentiation domains may vary, the differentiation domain of the second nucleic acid tag must be different than the differentiation domain of the first nucleic acid tag in this embodiment. The differentially tagged nucleic acids are mixed, amplified, and assessed by gel electrophoresis.

As with other examples in this specification, a nucleic acid target may be only one of a plurality of nucleic acid targets to be analyzed and the first and second populations may be two members of a plurality of populations being analyzed.

### Example 6

### Nucleic Fingerprint Analysis

Nucleic acid fingerprint analysis has been used extensively to identify genes that are differentially expressed between samples. Often fingerprint analysis produces a high rate of false positives. The number of false positives can be drastically reduced by using. population tagging to generate cDNA populations for arbitrarily primed PCR.

In an example of fingerprint analysis employing the aspects of the invention, two or more RNA samples are reverse transcribed with tags comprising anchored oligodT at their 3′ ends, a primer binding, transcription or affinity site as a differentiation domain, and a PCR primer binding site as an amplification domain. Differentially tagged cDNA populations are mixed and co-amplified using a primer specific to the PCR primer binding site of the tag and at least one arbitrary sequence primer. Following amplification, the PCR products are distinguished using the unique differentiation domains specific to each sample. The differentiated nucleic acids may be fractionated and analyzed by any methods known to those of skill. For example, they may be fractionated in adjacent lanes on a sequencing gel and the labeled products detected via autoradiography, with bands of differing intensity representing differentially expressed genes. These bands may be removed, cloned, and sequenced, if desired.

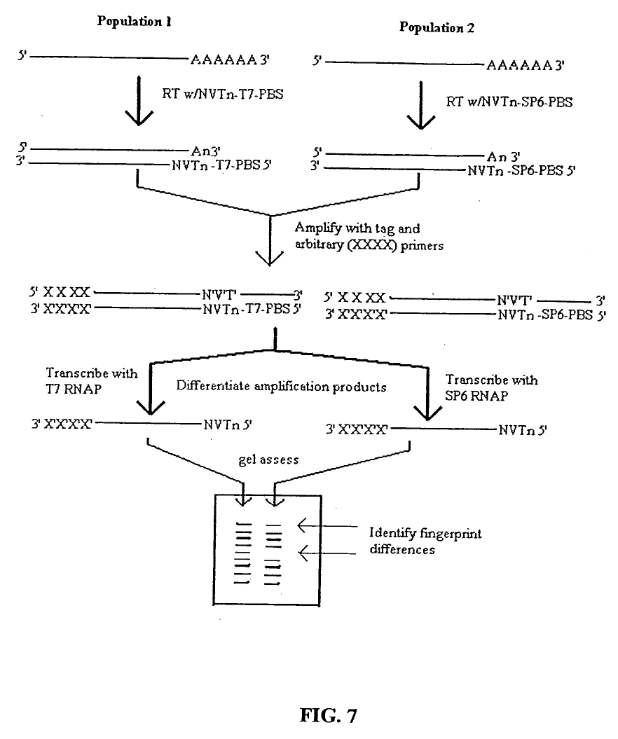

FIG. 7 depicts one specific embodiment of the invention, which compares a first RNA target within two or more populations. In this protocol, a first nucleic acid tag comprising anchored oligodT (i.e., NV polyT), an amplification domain (“A.D.” i.e., a primer binding site, PBS) and a differentiation domain comprising a first transcription domain (i.e., a T7 promoter) is appended (via reverse transcription) to the nucleic acids of a first sample. A second nucleic acid tag comprising anchored oligodT (i.e., NV polyT), an amplification site (i.e., a primer binding domain, PBS) and a differentiation domain comprising a second transcription domain (i.e., a SP6 promoter) is appended to the nucleic acids of a second sample. The first and second populations may be only two of plurality of populations being analyzed.

Each of the differentially tagged populations are mixed to provide a sample mixture. The tagged nucleic acids in the sample mixture are annealed to and co-amplified (e.g., via PCR) with one or more arbitrary primers (XXXX) and a tag specific (i.e., amplification domain specific) primer, producing a first amplified nucleic acid comprising a differentiation domain of the first nucleic acid tag and a nucleic acid segment of the first sample RNA or DNA, and a second amplified nucleic acid comprising a differentiation domain of the second nucleic acid tag and a nucleic acid segment of the second sample RNA or DNA (FIG. 7). The amplified nucleic acids are differentiated by transcription and the differentiated nucleic acids compared to determine the abundance (i.e. concentration) of the nucleic acids in the first population to the abundance of the nucleic acids in the second population.

This method of fingerprint analysis is superior to existing methods of differential display because the amplification is performed in a single tube. Thus any conditions that affect the amplification of any given target will affect its counterpart(s) in the other sample(s).

Techniques for use of the invention in regard to fingerprint analysis are further described in co-pending U.S. patent application Ser. No. 60/265,693, entitled “METHODS FOR NUCLEIC ACID FINGERPRINT ANALYSIS,” filed on Jan. 31, 2001, the disclosure of which is specifically incorporated herein by reference in it entirety without disclaimer.

### Example 7

### Tagged Array Analysis

Population tagging can also be used to convert RNA samples into labeled products for array analysis. Two or more populations can be tagged so that they share PCR primer binding sites but have distinct differentiation domains to support differential labeling. The tagged cDNAs can be mixed and amplified using a primer for the tags and a collection of primers specific to the mRNA targets that are being evaluated by the array. The amplified population can be split into labeling reactions specific to each differentiation domain to produce labeled, differentiated nucleic acids specific to each population. The labeled nucleic acids can then be assessed using existing array technology.

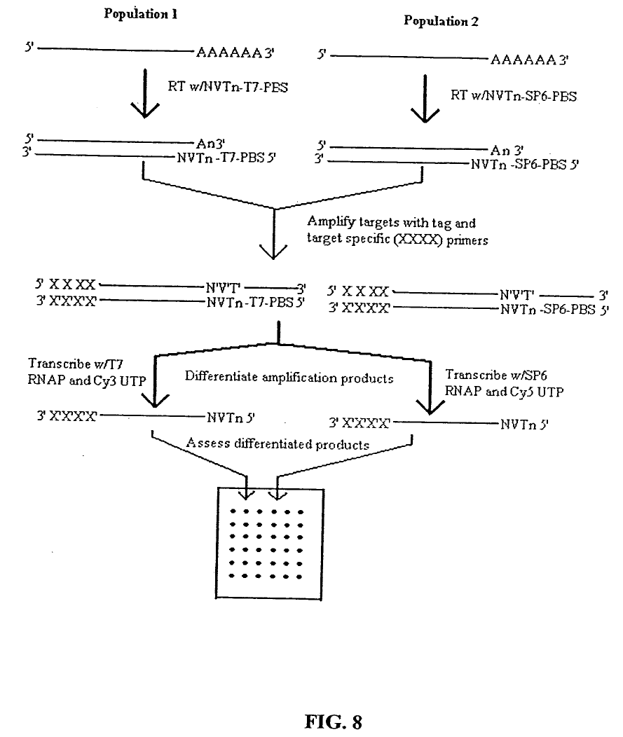

FIG. 8 illustrates a particular application of tagged array analysis. In the example, nucleic acid populations 1 and 2 are tagged by reverse transcription using primers with identical Primer Binding Sites (PBS) and a promoter for T7 or SP6 RNA polymerase. The differentially tagged cDNAs are mixed and targets are amplified by PCR using one primer specific to the PBS of the tag and a collection of primers specific to targets. The amplified sample is split into two transcription reactions, one with T7 RNA polymerase and Cy3 NTP and one with SP6 RNA polymerase and a Cy5 NTP. The labeled RNAs can then be hybridized to a single array.

This method is superior to existing methods of nucleic acid amplification for array analysis because the amplification is performed in a single tube. Thus any conditions that affect the amplification of any given target will affect its counterpart(s) in the other sample(s).

Techniques for use of the invention in regard to array analysis are further described in co-pending U.S. patent application Ser. No. 60/265,695, entitled “COMPETITIVE POPULATION NORMALIZATION FOR COMPARATIVE ANALYSIS OF NUCLEIC ACID SAMPLES,” filed on Jan. 31, 2001, the disclosure of which is specifically incorporated herein by reference in it entirety without disclaimer.

### Example 8

### Schematic for Massively Parallel Sample Analysis of Single Targets

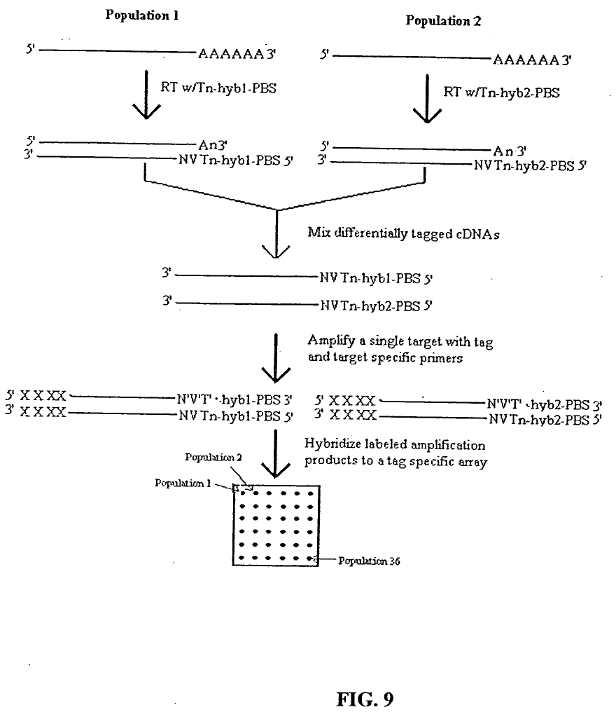

Another use of population tagging is measuring the relative abundance of a nucleic acid target in many different samples (FIG. 9). In one embodiment, unique affinity domains are used to differentially tag multiple RNA or DNA samples. The differentially tagged cDNAs are mixed and a single target present in the sample mixture is amplified using one primer specific to the amplification domain of the tag and one primer specific to the target. A labeled nucleotide or primer could be incorporated during the amplification reaction or the amplification products could be used in a subsequent labeling reaction (for instance, a transcription reaction) provided that an appropriate labeling domain is present in the tag sequences. The labeled nucleic acids are distinguished using ligands specific to each affinity domain present in the various tags. For instance, oligonucleotides specific to each affinity domain could be spotted at unique addresses on an array. The labeled products generated during or subsequent to target amplification could be hybridized to the array. The signal from each address on the array could be quantified to reveal the relative abundance of the target in each sample. FIG. 9 depicts one particular embodiment of this application of the invention.

Techniques for use of the invention in regard to this form of array analysis are further described in co-pending U.S. patent application Ser. No. 60/265,692, entitled “COMPETITIVE AMPLIFICATION OF FRACTIONATED TARGETS FROM MULTIPLE NUCLEIC ACID SAMPLES,” filed on Jan. 31, 2001, the disclosure of which is specifically incorporated herein by reference in it entirety without disclaimer.

All of the compositions and/or methods disclosed and claimed herein can be made and executed without undue experimentation in light of the present disclosure. While the compositions and methods of this invention have been described in terms of preferred embodiments, it are apparent to those of skill in the art that variations may be applied to the compositions and/or methods and in the steps or in the sequence of steps of the method described herein without departing from the concept, spirit and scope of the invention. More specifically, it are apparent that certain agents which are both chemically and physiologically related may be substituted for the agents described herein while the same or similar results are achieved. All such similar substitutes and modifications apparent to those skilled in the art are deemed to be within the spirit, scope and concept of the invention as defined by the appended claims.

References

The following references, to the extent that they provide exemplary procedural or other-details supplementary to those set forth herein, are specifically incorporated herein by reference.

Beasley et al., “Statistical Refinement of Primer Design Parameters,” In PCR Applications, Innis, Gelfand, Sninsky (Eds.), pp. 55-72, 1999.

Butler and Chamberlin, J. Biol. Chem., 257:5772-5778, 1982.

Chamberlin and Ryan, in The Enzymes, ed. P. Boyer Academic Press, New York, pp. 87-108, 1982.

Chapman and Burgess, Nucleic Acids Res. 16:5413, 1987.

Chapman and Wells, Nucleic Acids Res. 10(20):6331, 1982.

Chee et al, “Assessing genetic information with high density DNA Arrays,” Science, 274:610-614, 1996.

Compton J. “Nucleic acid sequence-based amplification,” Nature. 7;3 50(6313):91-92, 1991.

Diaz et al., J. Mol. Biol. 229: 805-811, 1993.

Duggan et al., “Expression profiling using cDNA microarrays,” Nat Genet. 21(1 Suppl):10-14, 1999.

Dunn and Studier, J. Mol. Biol. 166:477-535, 1983; and erratum J. Mol. Biol. 175:111-112, 1984.

Dunn et al., Nature New Biology, 230:94-96, 1971.

Egholm et al., “PNA hybridizes to complementary oligonucleotides obeying the Watson-Crick hydrogen-bonding rules,” Nature, 365(6446):566-568, 1993.

European Patent No. 266,032

European Patent No. 329 822

European Patent No. 98302726

Froehler et al., “Synthesis of DNA via deoxynucleoside H-phosphonate intermediates,” Nucleic Acids Res. 14(13):5399-5407, 1986.

GB Application No. 2 202 328

Guatelli et al., “Isothermal, in vitro amplification of nucleic acids by a multienzyme reaction modeled after retroviral replication,” Proc Natl Acad Sci U S A. 87(5): 1874-1878, 1990.

Hausmann, Current Topics in Microbiology and Immunology, 75:77-109, 1976.

Ikeda et al., “Selection and characterization of a mutant T7 RNA polymerase that recognizes an expanded range of T7 promoter-like sequences,” Biochemistry 32: 9115-9124, 1993.

Innis et al., “DNA sequencing with Thermus aquaticus DNA polymerase and direct sequencing of polymerase chain reaction-amplified DNA,” Proc Natl Acad Sci U S A. 85(24):9436-9440, 1988.

Kato, “Adaptor-tagged competitive-PCR: A novel method for measuring relative gene expression,” Nuc Acids Res 25:4694-4696, 1997.

Klement et al., J. Mol. Biol. 215:21-29, 1990.

Kornberg and Baker, DNA Replication, Second Edition, 1992, New York, W. H. Freeman and Company, 1992.

Korsten et al., J. Gen. Virol., 43:57-73, 1975.

Kotani et al. (1987), Nucl. Acids Res. 15:2653-2664, 1987.

Kwoh et al., “Transcription-based amplification system and detection of amplified human immunodeficiency virus type 1 with a bead-based sandwich hybridization format, Proc Natl Acad Sci U S A. 86(4):1173-1177, 1989.

Lizardi et al., “Mutation Detection and Single Molecule Counting Using Isothermal Rolling Circle Amplification,” Nat. Genetics 19:225-232, 1998.

Lizardi et al., Bio/technology 6:1197-1202, 1988.

Lockhart et al., “Expression monitoring by hybridization to high-density oligonucleotide arrays,” Nat Biotechnol. 14(13):1675-1680, 1996.

Lomeli et al., Clin. Chem. 35:1826-1831, 1989

Matoba, et al., Gene 241:125-131, 2000.

Morris et al., Gene 41:221-227, 1986.

PCT Application No. PCT/EP/01219

PCT Application No. PCT/US89/01025

PCT Application No. WO 88/10315

PCT Application No. WO 90/07641

PCT Application No. WO 92/20702

Phillips and Eberwine Methods: A Companion to Methods in Enzymology 10:283-288, 1996.

Sambrook et al., In: Molecular Cloning: A Laboratory Manual, Vol. 1, Cold Spring Harbor Laboratory Press, Cold Spring Harbor, N.Y., Ch. 7,7.19-17.29, 1989.

Scheit, Nucleotide Analogs, Synthesis and Biological Function, Wiley-Interscience, New York, pp. 171-172, 1980.

Schneider and Stormo, Nucleic Acids Res. 17(2):659, 1989.

Towle et al., J Biol. Chem., 250:1723-1733, 1975.

U.S. Pat. No. 4,659,774

U.S. Pat. No. 4,683,195

U.S. Pat. No. 4,683,202

U.S. Pat. No. 4,786,600

U.S. Pat. No. 4,800,159

U.S. Pat. No. 4,816,571

U.S. Pat. No. 4,952,496

U.S. Pat. No. 4,959,463

U.S. Pat. No. 5,141,813

U.S. Pat. No. 5,210,015

U.S. Pat. No. 5,214,136

U.S. Pat. No. 5,223,618

U.S. Pat. No. 5,262,311

U.S. Pat. No. 5,264,566

U.S. Pat. No. 5,279,721

U.S. Pat. No. 5,340,728

U.S. Pat. No. 5,378,825

U.S. Pat. No. 5,428,148

U.S. Pat. No. 5,446,137

U.S. Pat. No. 5,470,967

U.S. Pat. No. 5,487,993

U.S. Pat. No. 5,514,545

U.S. Pat. No. 5,539,082

U.S. Pat. No. 5,545,522

U.S. Pat. No. 5,554,744

U.S. Pat. No. 5,574,146

U.S. Pat. No. 5,602,240

U.S. Pat. No. 5,602,244

U.S. Pat. No. 5,610,289

U.S. Pat. No. 5,614,617

U.S. Pat. No. 5,623,070

U.S. Pat. No. 5,645,897

U.S. Pat. No. 5,652,099

U.S. Pat. No. 5,670,663

U.S. Pat. No. 5,672,697

U.S. Pat. No. 5,681,947

U.S. Pat. No. 5,695,937

U.S. Pat. No. 5,700,922

U.S. Pat. No. 5,705,629

U.S. Pat. No. 5,708,154

U.S. Pat. No. 5,712,126

U.S. Pat. No. 5,714,331

U.S. Pat. No. 5,714,606

U.S. Pat. No. 5,719,262

U.S. Pat. No. 5,736,336

U.S. Pat. No. 5,763,167

U.S. Pat. No. 5,766,855

U.S. Pat. No. 5,773,571

U.S. Pat. No. 5,777,092

U.S. Pat. No. 5,786,461

U.S. Pat. No. 5,792,847

U.S. Pat. No. 5,792,847

U.S. Pat. No. 5,824,528

U.S. Pat. No. 5,830,694

U.S. Pat. No. 5,840,873

U.S. Pat. No. 5,843,640

U.S. Pat. No. 5,843,650

U.S. Pat. No. 5,843,651

U.S. Pat. No. 5,846,708

U.S. Pat. No. 5,846,709

U.S. Pat. No. 5,846,717

U.S. Pat. No. 5,846,726

U.S. Pat. No. 5,846,729

U.S. Pat. No. 5,846,783

U.S. Pat. No. 5,849,487

U.S. Pat. No. 5,849,497

U.S. Pat. No. 5,849,546

U.S. Pat. No. 5,849,547

U.S. Pat. No. 5,853,990

U.S. Pat. No. 5,853,992

U.S. Pat. No. 5,853,993

U.S. Pat. No. 5,856,092

U.S. Pat. No. 5,858,652

U.S. Pat. No. 5,859,221

U.S. Pat. No. 5,861,244

U.S. Pat. No. 5,863,732

U.S. Pat. No. 5,863,753

U.S. Pat. No. 5,866,331

U.S. Pat. No. 5,866,366

U.S. Pat. No. 5,872,232

U.S. Pat. No. 5,882,864

U.S. Pat. No. 5,886,165

U.S. Pat. No. 5,891,625

U.S. Pat. No. 5,891,681

U.S. Pat. No. 5,905,024

U.S. Pat. No. 5,908,845

U.S. Pat. No. 5,910,407

U.S. Pat. No. 5,912,124

U.S. Pat. No. 5,912,145

U.S. Pat. No. 5,916,776

U.S. Pat. No. 5,919,630

U.S. Pat. No. 5,922,574

U.S. Pat. No. 5,925,517

U.S. Pat. No. 5,928,862

U.S. Pat. No. 5,928,869

U.S. Pat. No. 5,928,905

U.S. Pat. No. 5,928,906

U.S. Pat. No. 5,929,227

U.S. Pat. No. 5,932,413

U.S. Pat. No. 5,932,451

U.S. Pat. No. 5,935,791

U.S. Pat. No. 5,935,825

U.S. Pat. No. 5,939,291

U.S. Pat. No. 5,942,391

U.S. Pat. No. 5,962,271

U.S. Pat. No. 6,025,134

U.S. Pat. No. 6,037,130

U.S. Pat. No. 6,057,134

U.S. Pat. No. 6,107,037

Watson et al., Molecular Biology of The Gene, 4th Ed., Chapters 13-15, Benjamin/Cummings Publishing Co., Menlo Park, Calif.

Welsh and McClelland, Nuc. Acids Res 18:7213-7218, 1990.

Zhang et al., “Amplification of Target-Specific, Ligation-Dependent Circular Probe,” Gene 211:, 277-285, 1998.

1. A method of comparing one or more nucleic acid targets within two or more samples, comprising:
a) appending at least a first nucleic acid tag comprising a first amplification domain and a first differentiation domain to at least a first nucleic acid target of at least a first sample, wherein the first differentiation domain comprises a first primer binding domain, and wherein the differentiation domain of the first tag is appended between the first nucleic acid target sequence and the amplification domain;
b) appending at least a second nucleic acid tag comprising a second amplification domain and a second differentiation domain to at least the first nucleic acid target of at least a second sample, wherein the second differentiation domain comprises a second primer binding domain that is different than the first primer binding domain, and wherein the differentiation domain of the second tag is appended between the at least a first nucleic acid target sequence and the amplification domain;
c) co-amplifying the first nucleic acid target of the first sample and the first nucleic acid target of the second sample, wherein the amplifying produces at least a first amplified nucleic acid comprising at least the first primer binding domain and a segment of the target nucleic acid and a second amplified nucleic acid comprising at least the second primer binding domain and a segment of the target nucleic acid from the second sample;
d) differentiating the first amplified nucleic acid, wherein the differentiating comprises annealing at least a first differentiation primer to the first primer binding domain, wherein the differentiating further comprises extension of the first differentiation primer to produce at least a first differentiated nucleic acid;
e) differentiating the second amplified nucleic acid, wherein the differentiating further comprises annealing at least a second differentiation primer to the second primer binding domain, wherein the differentiating further comprises extension of the second differentiation primer to produce at least a second differentiated nucleic acid; and
f) comparing abundance of the differentiated nucleic acid from the first nucleic acid target of the first sample to abundance of the differentiated nucleic acid from the first nucleic acid target of the second sample.

2. The method of claim 1, wherein said first differentiated nucleic acid or the second differentiated nucleic acid includes a detectable moeity.

3. A method of comparing one or more single-stranded nucleic acid targets within two or more samples, comprising:
a) obtaining at least a first sample and a second sample, each potentially having at least a first nucleic acid target;
b) preparing at least a first tagged nucleic acid sample by appending at least a first nucleic acid tag comprising a first amplification domain and a first differentiation domain to the first nucleic acid target of the first sample, if the first nucleic acid target is present in the first sample;
c) preparing at least a second tagged nucleic acid sample by appending at least a second nucleic acid tag comprising a second amplification domain and a second differentiation domain to the first nucleic acid target of the second sample, if the first nucleic acid target is present in the second sample;
d) mixing the first tagged nucleic acid sample and the second tagged nucleic acid sample to create a sample mixture;
e) co-amplifying said first nucleic acid target of the first sample and said first nucleic acid target of the second sample in the sample mixture, if both the first and second nucelic acid targets are present in the sample mixture, wherein said co-amplifying produces at least a first amplified nucleic acid comprising at least the first differentiation domain and a segment of the target nucleic acid from the first sample, if the first nucleic acid target is present in the first sample, and at least a second amplified nucleic acid comprising at least the second differentiation domain and a segment of the target nucleic acid from the second sample, if the first nucleic acid target is present in the second sample;
f) differentiating the first amplified nucleic acid, if any, from the second amplified nucleic acid, if any; and
g) comparing abundance of the differentiated nucleic acid from the first nucleic acid target of said first sample to abundance of the differentiated nucleic acid from the first nucleic acid target of said second sample.

4. The method of claim 3, wherein the first nucleic acid target is present in the first sample.

5. The method of claim 4, wherein the first nucleic acid target is present in the second sample.

6. The method of claim 3, wherein the differentiation domain of the first tag and the second tag is appended between the first nucleic acid target sequence and the amplification domain.

7. The method of claim 3, wherein said nucleic acid target is one target of a plurality of nucleic acid targets within the samples.

8. The method of claim 3, wherein said first and second sample are two samples of a plurality of samples.

9. The method of claim 8, wherein the first and second tag are two tags of a plurality of tags.

10. The method of claim 3, wherein the amplification domain of the first nucleic acid tag and the second nucleic acid tag comprises a primer binding domain.

11. The method of claim 3, wherein the amplification domain of the first nucleic acid tag and the second nucleic acid tag comprises a transcription domain.

12. The method of claim 3, wherein the amplification domains of the first and second nucleic acid tags are functionally equivalent.

13. The method of claim 12, wherein the amplification domains of the first and second. nucleic acid tags are identical.

14. The method of claim 3, wherein the differentiation domain of the first nucleic acid tag and the second nucleic acid tag comprise at least a primer binding domain, a transcription domain, a size differentiation domain, an affinity domain, a unique sequence domain, or a restriction enzyme domain.

15. The method of claim 3, wherein differentiating comprises production of at least one differentiated nucleic acid from said first or second amplified nucleic acid.

16. The method of claim 15, wherein said differentiated nucleic acid is labeled in a detectable manner.

17. The method of claim 3, wherein said differentiation domains of the first nucleic acid tag and the second nucleic acid tag are affinity domains.

18. The method of claim 17, wherein differentiating comprises binding at least a first ligand to at least a segment of the affinity domain.

19. The method of claim 18, wherein the first ligand comprises a nucleic acid.

20. The method of claim 18, wherein the first ligand is bound to a solid support.

21. The method of claim 20, wherein the first ligand is used to separate the first target nucleic acid from at least one other nucleic acid or molecule.

22. The method of claim 20, wherein the solid support is a membrane, a bead, a glass slide, or a microtiter well.

23. The method of claim 20, wherein the amplified nucleic acid is labeled in a detectable manner.

24. The method of claim 18, wherein the first ligand is labeled.

25. The method of claim 24, wherein binding of the first ligand to said segment of the affinity domain results in a detectable signal.

26. The method of claim 3, wherein said differentiation domain of the first nucleic acid tag and the differentiation domain of the second nucleic acid tag are primer binding domains.

27. The method of claim 26, wherein. differentiating comprises binding at least a first differentiation primer to at least one segment of the primer binding domain.

28. The method of claim 27, further comprising at least one primer extension reaction.

29. The method of claim 28, wherein said primer extension reaction produces at least one differentiated nucleic acid.

30. The method of claim 29, wherein said differentiated nucleic acid is labeled with a detectable moiety.

31. The method of claim 3, wherein said differentiation domains of the first and second nucleic acids are unique sequence domains.

32. The method of claim 31, wherein differentiating comprises sequencing through the differentiation domains of the amplified nucleic acids.

33. The method of claim 3, wherein the differentiation domains of the first nucleic acid tag and the second nucleic acid tag each comprise at least one transcription domain.

34. The method of claim 33, wherein said differentiation domain comprises a promoter for a prokaryotic RNA polymerase.

35. The method of claim 33, wherein differentiating comprises a transcription reaction.

36. The method of claim 35, wherein said transcription reaction produces at least one differentiated nucleic acid.

37. The method of claim 36, wherein said differentiated nucleic acid includes a detectable moiety.

38. The method of claim 3, wherein the differentiation domain of the first nucleic acid tag and the second nucleic acid tag each comprise at least one size differentiation domain.

39. The method of claim 38, wherein said differentiating comprises distinguishing the amplification products from the first and second samples by size.

40. The method of claim 3, wherein said differentiation domain of the first nucleic acid tag or the second nucleic acid tag comprises at least one restriction enzyme cleavage domain.

41. The method of claim 40, further comprising cleaving said restriction enzyme cleavage site to promote the ligation of a label or at least one additional domain to a segment of the at least a first or at least a second nucleic acid tag.

42. The method of claim 40, wherein differentiating comprises cleaving said restriction enzyme site to remove at least one label.

43. The method of claim 3, wherein the first nucleic acid tag or the second nucleic acid tag further comprises at least one additional domain.

44. The method of claim 43, wherein said additional domain is labeling domain, a restriction enzyme domain, a secondary amplification domain, a secondary differentiation domain or a sequencing primer binding domain.

45. The method of claim 43, wherein said additional domain comprises at least one labeling domain.

46. The method of claim 45, wherein said labeling domain is comprised between the differentiation domain and the amplification domain.

47. A method of comparing one or more nucleic acid targets within two or more samples, comprising:
a) appending at least a first nucleic acid tag comprising at least a first amplification domain and at least a first differentiation domain to at least a first nucleic acid target of at least a first sample, wherein said first differentiation domain comprises at least one affinity domain, primer binding domain, or transcription domain;
b) appending at least a second nucleic acid tag comprising at least a second amplification domain and at least a second differentiation domain to the first nucleic acid target of at least a second sample, wherein the second differentiation domain is different than the first differentiation domain and comprises at least one affinity domain, primer binding domain, or transcription domain;
c) co-amplifying said first nucleic acid target of the first sample and said first nucleic acid target of the second sample, wherein said amplifying produces at least a first amplified nucleic acid comprising at least the first differentiation domain and a segment of the target nucleic acid from the first sample and at least a second amplified nucleic acid comprising at least the second differentiation domain and a segment of the target nucleic acid from the second sample;
d) differentiating the first amplified nucleic acid from the second amplified nucleic acid; and
e) comparing abundance of the differentiated nucleic acid from the first nucleic acid target of said first sample to abundance of the differentiated nucleic acid from the first nucleic acid target of said second sample.

48. A method of comparing one or more nucleic acid targets within two or more samples, comprising:
a) appending at least a first nucleic acid tag comprising a first amplification domain and a first differentiation domain to at least a first nucleic acid target of at least a first sample, wherein the first differentiation domain comprises a first transcription domain, and wherein the differentiation domain of the first tag is appended between the first nucleic acid target sequence and the amplification domain;
b) appending at least a second nucleic acid tag comprising a second amplification domain and a second differentiation domain to the first nucleic acid target of at least a second sample, wherein the second differentiation domain comprises a second transcription domain that is different than the first transcription domain, and wherein the differentiation domain of the second tag is appended between the at least a first nucleic acid target sequence and the amplification domain;
c) co-amplifying the first nucleic acid target of the first sample and the first nucleic acid target of the second sample, wherein the amplifying produces at least a first amplified nucleic acid comprising the at least first transcription domain and a segment of the target nucleic acid from the first sample and a second amplified nucleic acid comprising at least the second transcription domain and a segment of the target nucleic acid from the second sample;
d) differentiating the first amplified nucleic acid, wherein the differentiating comprises transcription from the first transcription domain to produce at least a first differentiated nucleic acid;
e) differentiating the second amplified nucleic acid, wherein the differentiating further comprises transcription from the second transcription domain to produce at least a second differentiated nucleic acid; and
f) comparing abundance of the differentiated nucleic acid from the first nucleic acid target of said first sample to abundance of the differentiated nucleic acid from the first nucleic acid target of said second sample.

49. The method of claim 48, wherein each of the first and second differentiated nucleic acids comprise at least one detectable moeity.

50. A method of comparing one or more single-stranded nucleic acid targets within two or more samples, comprising:
a) appending at least a first single-stranded nucleic acid tag comprising a first amplification domain and a first differentiation domain to at least a first nucleic acid target of at least a first sample, wherein the first differentiation domain comprises a first size differentiation domain, and wherein the differentiation domain of the first tag is appended between the first nucleic acid target sequence and the amplification domain;
b) appending at least a second single-stranded nucleic acid tag comprising a second amplification domain and a second differentiation domain to the first nucleic acid target of at least a second sample, wherein the second differentiation domain comprises a second size differentiation domain that is different than the first size differentiation domain, and wherein the differentiation domain of the second tag is appended between the at least a first nucleic acid target sequence and the amplification domain;
c) co-amplifying the first nucleic acid target of the first sample and the first nucleic acid target of the second sample, wherein the co-amplifying produces at least a first amplified nucleic acid comprising at least the first size differentiation domain and a segment of the target nucleic acid and a second amplified nucleic acid comprising at least the second size differentiation domain and a segment of the target nucleic acid;
d) differentiating the first amplified nucleic acid, wherein said differentiating comprises determining the electrophoretic mobility of the first amplified nucleic acid;
e) differentiating the second amplified nucleic acid, wherein said differentiating further comprises determining the electrophoretic mobility of the second amplified nucleic acid; and
f) comparing abundance of the differentiated nucleic acid from the first nucleic acid target of said first sample to abundance of the differentiated nucleic acid from the first nucleic acid target of said second sample.

51. A method of comparing one or more nucleic acid targets within two or more samples, comprising:
a) appending at least a first nucleic acid tag comprising a first amplification domain and a first differentiation domain to at least a first nucleic acid target of at least a first sample, wherein the first differentiation domain comprises a first affinity domain, and wherein the differentiation domain of the first tag is appended between the first nucleic acid target sequence and the amplification domain;
b) appending at least a second nucleic acid tag comprising a second amplification domain and a second differentiation domain to the first nucleic acid target of at least a second sample, wherein the second differentiation domain comprises a second affinity domain that is different than the first affinity domain, and wherein the differentiation domain of the second tag is appended between the at least a first nucleic acid target sequence and the amplification domain;
c) co-amplifying the first nucleic acid target of the first sample and the first nucleic acid target of the second sample to produce at least a first amplified nucleic acid comprising at least the first affinity domain and a segment of the target nucleic acid from the first sample and a second amplified nucleic acid comprising at least the second affinity domain and a segment of the target nucleic acid from the second sample;
d) differentiating the first amplified nucleic acid, wherein the differentiating comprises binding of the first amplified nucleic acid to an at least a first ligand;
f) differentiating the second amplified nucleic acid, wherein the differentiating further comprises binding of the second amplified nucleic acid to an at least a second ligand; and
g) comparing abundance of the differentiated nucleic acid from the first nucleic acid target of said first sample to abundance of the differentiated nucleic acid from the first nucleic acid target of said second sample.
